(19) United States
(12) Patent Application Publication
(10) Pub. No.: US 2025/0034533 A1
(43) Pub. Date: Jan. 30, 2025

(54) Compositions And Methods For Treating Mucopolysaccharidosis IIIA

(71) Applicant: Amicus Therapeutics, Inc, Philadelphia, PA (US)

(72) Inventors: Leida Rassouli-Taylor, Philadelphia, PA (US); Hung V. Do, New Hope, PA (US); Steven Tuske, Cranbury, NJ (US); Nastry Brignol, Philadelphia, PA (US)

(73) Assignee: Amicus Therapeutics, Inc., Philadelphia, PA (US)

(21) Appl. No.: 18/709,210

(22) PCT Filed: Nov. 11, 2022
(86) PCT No.: PCT/US2022/079716

§ 371 Date: May 10, 2024

Related U.S. Application Data
(60) Provisional application No. 63/278,626, filed on Nov. 12, 2021.

(51) Int. Cl. C12N 9/14; C12N 15/86; A61K 38/00

(52) U.S. Cl. CPC C12N 9/14; C12N 15/86; C12Y 310/01001; A61K 38/00; C12N 2750/14143

(57) ABSTRACT

Provided herein are compositions and methods for treating Mucopolysaccharidosis IIIA (MPS IIIA) comprising enzyme replacement therapy and gene therapy constructs described herein.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AMCS_002_00US_SeqList_ST25.txt, date recorded: Nov. 12, 2021, file size ˜136,142 bytes).

### TECHNICAL FIELD

The disclosure relates to a lysosomal enzyme variants and methods of using the same.

### BACKGROUND

Mucopolysaccharidosis IIIA (MPS IIIA, Sanfilippo Syndrome Type A) is a rare genetic disorder caused by a deficiency in N-sulfoglucosamine sulfohydrolase (SGSH), a lysosomal enzyme that degrades heparan sulfate, a type of glycosaminoglycan (GAG). SGSH deficiency leads to accumulation of undegraded heparan sulfate, damaging cells and tissues, and leading to organ dysfunction. MPS IIIA is characterized by severe central nervous system degeneration and progressive developmental delay with onset between 2 and 6 years with survival only until the second or third decade of life. There is no approved treatment for MPS IIIA.

### SUMMARY

Provided herein are compositions and methods for treating MPS IIIA, including novel SGSH variants for use in gene therapy or enzyme replacement therapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows the Rosetta energy scores calculated for several SGSH mutants. The energy and structure of each mutant was calculated at least ten times, in order to achieve a statistical spread of values representing each mutant. The Rosetta energy score calculated for each individual structure is represented by a circle. The error bars indicate the mean energy score and standard deviation for each mutant. The energy scores for single mutants A482Y and E488V, as well as the A482Y/E488V double mutant are significantly lower than those for the WT protein (unpaired t-test, p<0.0001).

FIG. 2A shows an anti-SGSH western blot showing levels of different mutants of SGSH in conditioned media. Bands on right half of gel appear to run higher because the gel tore and was repositioned imperfectly. “Quad” refers to the quadruple mutant containing the following mutations: Y89F, V95W, N99W, D102P. “Mock” refers to the empty vector control transfection.

FIG. 2B shows western blot quantification of SGSH mutants as compared with WT SGSH in conditioned medium of transfected Freestyle 293 cells.

FIG. 3 shows SGSH activity in conditioned medium harvested from Freestyle 293F cells transfected with different SGSH constructs. Fluorescence activity assay performed in duplicate. Mutants of interest retain their enzymatic activity.

FIG. 4A-FIG. 4B show a Western blot and quantification of SGSH in conditioned medium of transfected Freestyle 293 cells. Comparison of SGSH mutants expressed with and without C-terminal vIGF2 fusion. Tagging with vIGF2 does not appreciably decrease SGSH levels secreted into the media, for the constructs tested.

FIG. 5 shows binding of SGSH variants to CIMPR in in vitro plate-binding assay. SGSH A482Y E488V double mutation does not affect binding to CIMPR. vIGF2-tagged SGSH variants show significantly increased binding to CIMPR.

FIG. 6 shows SGSH activity and GAG (HS) levels in the brain. Highest activity is achieved in the hSGSH treatment group, while greatest GAG reduction is seen in the BiP-SGSH (stabilized) and BiP-SGSH-vIGF2 (stabilized) groups. Brown-Forsythe and Welch ANOVA tests, with Dunnett's T3 multiple comparisons test. *p<0.05. ****p<0001.

FIG. 7 shows SGSH activity and GAG (HS) levels in the spinal cord. Highest activity is achieved in the treatment groups that were not treated with vIGF2-tagged constructs. Significant GAG reduction was seen in all treatment groups except for low dose BiP-SGSH-vIGF2; all treatment groups saw significant (p<0.0001) reduction at the high dose. Brown-Forsythe and Welch ANOVA tests, with Dunnett's T3 multiple comparisons test.

FIG. 8 shows SGSH activity and GAG (HS) levels in the liver. Highest activity is achieved in the treatment groups treated with hSGSH and BiP-SGSH. Significant GAG reduction was seen in all treatment groups (p<0.001) except for low dose BiP-SGSH-vIGF2. Brown-Forsythe and Welch ANOVA tests, with Dunnett's T3 multiple comparisons test.

FIG. 9 shows SGSH activity in serum and plasma at day 7 and at day 28.

FIG. 10 shows anti-SGSH antibody titer in mouse plasma at day 28.

### DETAILED DESCRIPTION

### Variant SGSH Proteins

Provided herein are therapeutic human N-sulfoglucosamine sulfohydrolase (SGSH) variant proteins having at least one mutation that enhances stability and/or expression of the SGSH variant protein compared to wildtype (WT) human SGSH (SEQ ID NO:1). In some embodiments, the variant human SGSH proteins further comprise a variant human IGF2 (vIGF2) peptide at the N-terminus or the C-terminus of the therapeutic protein. The vIGF2 peptide improves cell uptake and lysosomal targeting of the variant SGSH protein. Examples of novel SGSH variant amino acid sequences are provided in Table 1 below.

TABLE 1
SGSH Amino Acid Sequences

| Name | Sequence | SEQ ID NO: |
|---|---|---|
| WTSGSH | MSCPVPACCALLLVLGLCRARPRNALLLLADDGGFESGAYNNSAIATP | 1 |
| (NCBI Reference | HLDALARRSLLFRNAFTSVSSCSPSRASLLTGLPQHQNGMYGLHQDVH | |
| Sequence: | HFNSFDKVRSLPLLLSQAGVRTGIIGKKHVGPETVYPFDFAYTEENGS | |
| NP_000190.1) | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIE | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CAPDGVLEEKLSPQCQPLHNEL | |
| SGSH | MSCPVPACCALLLVLGLCRARPRNALLLLADDGGFESGAYNNSAIATP | 2 |
| A482Y | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CYPDGVLEEKLSPQCQPLHNEL | |
| SGSH E488V | MSCPVPACCALLLVLGLCRARPRNALLLLADDGGFESGAYNNSAIATP | 3 |
| | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CAPDGVLVEKLSPQCQPLHNEL | |
| SGSH | MSCPVPACCALLLVLGLCRARPRNALLLLADDGGFESGAYNNSAIATP | 4 |
| A482Y/E488V | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CYPDGVLVEKLSPQCQPLHNEL | |
| BiP-SGSH | MKLSLVAAMLLLLSAARARPRNALLLLADDGGFESGAYNNSAIATPHL | 5 |
| | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCA | |
| | PDGVLEEKLSPQCQPLHNEL | |
| BiP-SGSH | MKLSLVAAMLLLLSAARARPRNALLLLADDGGFESGAYNNSAIATPHL | 6 |
| A482Y | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCY | |
| | PDGVLEEKLSPQCQPLHNEL | |
| BiP-SGSH | MKLSLVAAMLLLLSAARARPRNALLLLADDGGFESGAYNNSAIATPHL | 7 |
| E488V | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCA | |
| | PDGVLVEKLSPQCQPLHNEL | |
| BiP-SGSH | MKLSLVAAMLLLLSAARARPRNALLLLADDGGFESGAYNNSAIATPHL | 8 |
| A482Y/E488V | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCY | |
| | PDGVLVEKLSPQCQPLHNEL | |
| BiP-SGSH- | MKLSLVAAMLLLLSAARARPRNALLLLADDGGFESGAYNNSAIATPHL | 9 |
| vIGF2 | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCA | |
| | PDGVLEEKLSPQCQPLHNELRPRAVPTQGGGGSGGGGSSRTLCGGELV | |
| | DTLQFVCGDRGFLFSRPASRVSRRSRGIVEECCFRSCDLALLETYCAT | |
| | PARSE | |
| SGSH-vIGF2 | MSCPVPACCALLLVLGLCRARPRNALLLLADDGGFESGAYNNSAIATP | 10 |
| | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CAPDGVLEEKLSPQCQPLHNELRPRAVPTQGGGGSGGGGSSRTLCGGE | |
| | LVDTLQFVCGDRGELFSRPASRVSRRSRGIVEECCFRSCDLALLETYC | |
| | ATPARSE | |
| SGSH | MSCPVPACCALLLVLGLCRARPRNALLLLADDGGFESGAYNNSAIATP | 11 |
| A482YvIGF2 | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CYPDGVLEEKLSPQCQPLHNELRPRAVPTQGGGGSGGGGSSRTLCGGE | |
| | LVDTLQFVCGDRGFLFSRPASRVSRRSRGIVEECCFRSCDLALLETYC | |
| | ATPARSE | |
| SGSH E488V | MSCPVPACCALLLVLGLCRARPRNALLLLADDGGFESGAYNNSAIATP | 12 |
| vIGF2 | HLDALARRSLLFRNAFTSVSSCSPSRASLLTGLPQHQNGMYGLHQDVH | |
| | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CAPDGVLVEKLSPQCQPLHNELRPRAVPTQGGGGSGGGGSSRTLCGGE | |
| | LVDTLQFVCGDRGFLFSRPASRVSRRSRGIVEECCFRSCDLALLETYC | |
| | ATPARSE | |
| SGSH | MSCPVPACCALLLVLGLCRARPRNALLLLADDGGFESGAYNNSAIATP | 13 |
| A482Y/E488V | HLDALARRSLLFRNAFTSVSSCSPSRASLLTGLPQHQNGMYGLHQDVH | |
| vIGF2 | HFNSFDKVRSLPLLLSQAGVRTGIIGKKHVGPETVYPFDFAYTEENGS | |
| | VLQVGRNITRIKLLVRKFLQTQDDRPFFLYVAFHDPHRCGHSQPQYGT | |
| | FCEKFGNGESGMGRIPDWTPQAYDPLDVLVPYFVPNTPAARADLAAQY | |
| | TTVGRMDQGVGLVLQELRDAGVLNDTLVIFTSDNGIPFPSGRTNLYWP | |
| | GTAEPLLVSSPEHPKRWGQVSEAYVSLLDLTPTILDWFSIPYPSYAIF | |
| | GSKTIHLTGRSLLPALEAEPLWATVFGSQSHHEVTMSYPMRSVQHRHF | |
| | RLVHNLNFKMPFPIDQDFYVSPTFQDLLNRTTAGQPTGWYKDLRHYYY | |
| | RARWELYDRSRDPHETQNLATDPRFAQLLEMLRDQLAKWQWETHDPWV | |
| | CYPDGVLVEKLSPQCQPLHNELRPRAVPTQGGGGSGGGGSSRTLCGGE | |
| | LVDTLQFVCGDRGFLFSRPASRVSRRSRGIVEECCFRSCDLALLETYC | |
| | ATPARSE | |
| BiP-SGSH | MKLSLVAAMLLLLSAARARPRNALLLLADDGGFESGAYNNSAIATPHL | 14 |
| A482Y-vIGF2 | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCY | |
| | PDGVLEEKLSPQCQPLHNELRPRAVPTQGGGGSGGGGSSRTLCGGELV | |
| | DTLQFVCGDRGFLFSRPASRVSRRSRGIVEECCFRSCDLALLETYCAT | |
| | PARSE | |
| BiP-SGSH | MKLSLVAAMLLLLSAARARPRNALLLLADDGGFESGAYNNSAIATPHL | 15 |
| E488V-vIGF2 | DALARRSLLFRNAFTSVSSCSPSRASLLTGLPQHQNGMYGLHQDVHHF | |
| | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCA | |
| | PDGVLVEKLSPQCQPLHNELRPRAVPTQGGGGSGGGGSSRTLCGGELV | |
| | DTLQFVCGDRGFLFSRPASRVSRRSRGIVEECCFRSCDLALLETYCAT | |
| | PARSE | |
| BiP-SGSH | MKLSLVAAMLLLLSAARARPRNALLLLADDGGFESGAYNNSAIATPHL | 16 |
| A482Y/E488V- | DALARRSLLFRNAFTSVSSCSPSRASLLTGLPQHQNGMYGLHQDVHHF | |
| vIGF2 | NSFDKVRSLPLLLSQAGVRTGIIGKKHVGPETVYPFDFAYTEENGSVL | |
| | QVGRNITRIKLLVRKFLQTQDDRPFFLYVAFHDPHRCGHSQPQYGTFC | |
| | EKFGNGESGMGRIPDWTPQAYDPLDVLVPYFVPNTPAARADLAAQYTT | |
| | VGRMDQGVGLVLQELRDAGVLNDTLVIFTSDNGIPFPSGRTNLYWPGT | |
| | AEPLLVSSPEHPKRWGQVSEAYVSLLDLTPTILDWFSIPYPSYAIFGS | |
| | KTIHLTGRSLLPALEAEPLWATVFGSQSHHEVTMSYPMRSVQHRHFRL | |
| | VHNLNFKMPFPIDQDFYVSPTFQDLLNRTTAGQPTGWYKDLRHYYYRA | |
| | RWELYDRSRDPHETQNLATDPRFAQLLEMLRDQLAKWQWETHDPWVCY | |
| | PDGVLVEKLSPQCQPLHNELRPRAVPTQGGGGSGGGGSSRTLCGGELV | |
| | DTLQFVCGDRGFLFSRPASRVSRRSRGIVEECCFRSCDLALLETYCAT | |
| | PARSE | |
| Human SGSH | RPRNALLLLADDGGFESGAYNNSAIATPHLDALARRSLLFRNAFTSVS | 17 |
| Isoform 1, | SCSPSRASLLTGLPQHQNGMYGLHQDVHHFNSFDKVRSLPLLLSQAGV | |
| (residues 21-502) | RTGIIGKKHVGPETVYPFDFAYTEENGSVLQVGRNITRIKLLVRKFLQ | |
| Δ Signal | TQDDRPFFLYVAFHDPHRCGHSQPQYGTFCEKFGNGESGMGRIPDWTP | |
| Sequence | QAYDPLDVLVPYFVPNTPAARADLAAQYTTVGRMDQGVGLVLQELRDA | |
| | GVLNDTLVIFTSDNGIPFPSGRTNLYWPGTAEPLLVSSPEHPKRWGQV | |
| | SEAYVSLLDLTPTILDWFSIPYPSYAIFGSKTIHLTGRSLLPALEAEP | |
| | LWATVFGSQSHHEVTMSYPMRSVQHRHFRLVHNLNFKMPFPIDQDFYV | |
| | SPTFQDLLNRTTAGQPTGWYKDLRHYYYRARWELYDRSRDPHETQNLA | |
| | TDPRFAQLLEMLRDQLAKWQWETHDPWVCAPDGVLEEKLSPQCQPLHN | |
| | EL | |
| Human SGSH | RPRNALLLLADDGGFESGAYNNSAIATPHLDALARRSLLFRNAFTSVS | 18 |
| A482Y Δ Signal | SCSPSRASLLTGLPQHQNGMYGLHQDVHHFNSFDKVRSLPLLLSQAGV | |
| Sequence | RTGIIGKKHVGPETVYPFDFAYTEENGSVLQVGRNITRIKLLVRKFLQ | |
| | TQDDRPFFLYVAFHDPHRCGHSQPQYGTFCEKFGNGESGMGRIPDWTP | |
| | QAYDPLDVLVPYFVPNTPAARADLAAQYTTVGRMDQGVGLVLQELRDA | |
| | GVLNDTLVIFTSDNGIPFPSGRTNLYWPGTAEPLLVSSPEHPKRWGQV | |
| | SEAYVSLLDLTPTILDWFSIPYPSYAIFGSKTIHLTGRSLLPALEAEP | |
| | LWATVFGSQSHHEVTMSYPMRSVQHRHFRLVHNLNFKMPFPIDQDFYV | |
| | SPTFQDLLNRTTAGQPTGWYKDLRHYYYRARWELYDRSRDPHETQNLA | |
| | TDPRFAQLLEMLRDQLAKWQWETHDPWVCYPDGVLEEKLSPQCQPLHN | |
| | EL | |
| SGSH E488V Δ | RPRNALLLLADDGGFESGAYNNSAIATPHLDALARRSLLFRNAFTSVS | 19 |
| Signal Sequence | SCSPSRASLLTGLPQHQNGMYGLHQDVHHFNSFDKVRSLPLLLSQAGV | |
| | RTGIIGKKHVGPETVYPFDFAYTEENGSVLQVGRNITRIKLLVRKFLQ | |
| | TQDDRPFFLYVAFHDPHRCGHSQPQYGTFCEKFGNGESGMGRIPDWTP | |
| | QAYDPLDVLVPYFVPNTPAARADLAAQYTTVGRMDQGVGLVLQELRDA | |
| | GVLNDTLVIFTSDNGIPFPSGRTNLYWPGTAEPLLVSSPEHPKRWGQV | |
| | SEAYVSLLDLTPTILDWESIPYPSYAIFGSKTIHLTGRSLLPALEAEP | |
| | LWATVFGSQSHHEVTMSYPMRSVQHRHFRLVHNLNFKMPFPIDQDFYV | |
| | SPTFQDLLNRTTAGQPTGWYKDLRHYYYRARWELYDRSRDPHETQNLA | |
| | TDPRFAQLLEMLRDQLAKWQWETHDPWVCAPDGVLVEKLSPQCQPLHN | |
| | EL | |
| SGSH | RPRNALLLLADDGGFESGAYNNSAIATPHLDALARRSLLFRNAFTSVS | 20 |
| A482Y/E488V Δ | SCSPSRASLLTGLPQHQNGMYGLHQDVHHFNSFDKVRSLPLLLSQAGV | |
| Signal Sequence | RTGIIGKKHVGPETVYPFDFAYTEENGSVLQVGRNITRIKLLVRKFLQ | |
| | TQDDRPFFLYVAFHDPHRCGHSQPQYGTFCEKFGNGESGMGRIPDWTP | |
| | QAYDPLDVLVPYFVPNTPAARADLAAQYTTVGRMDQGVGLVLQELRDA | |
| | GVLNDTLVIFTSDNGIPFPSGRTNLYWPGTAEPLLVSSPEHPKRWGQV | |
| | SEAYVSLLDLTPTILDWFSIPYPSYAIFGSKTIHLTGRSLLPALEAEP | |
| | LWATVFGSQSHHEVTMSYPMRSVQHRHFRLVHNLNFKMPFPIDQDFYV | |
| | SPTFQDLLNRTTAGQPTGWYKDLRHYYYRARWELYDRSRDPHETQNLA | |
| | TDPRFAQLLEMLRDQLAKWQWETHDPWVCYPDGVLVEKLSPQCQPLHN | |
| | EL | |

In some embodiments, the variant SGSH protein comprises the A482Y mutation (i.e., the alanine residue at position 482 in the wildtype SGSH protein sequence is replaced with a tyrosine residue). In some embodiments, the variant SGSH protein comprises the E488V mutation. In some embodiments, variant SGSH protein comprises the A482Y mutation and the E488V mutation.

In some embodiments, the variant SGSH protein comprises an amino acid sequence that is at least 95% or at least 98% identical to SEQ ID NO: 2. In some embodiments, the variant SGSH protein comprises the amino acid sequence of SEQ ID NO: 2.

In some embodiments, the variant SGSH protein comprises an amino acid sequence that is at least 95% or at least 98% identical to SEQ ID NO: 3. In some embodiments, the variant SGSH protein comprises the amino acid sequence of SEQ ID NO: 3.

In some embodiments, the variant SGSH protein comprises an amino acid sequence that is at least 95% or at least 98% identical to SEQ ID NO: 4. In some embodiments, the variant SGSH protein comprises the amino acid sequence of SEQ ID NO: 4.

Further provided herein are nucleic acid sequences encoding novel SGSH variants. Such nucleic acid sequences include nucleic acid sequences encoding the amino acid sequence in Table 1 (SEQ ID NOs: 2-20). The nucleic acid sequences encoding novel SGSH variants can be included in expression vectors, such as plasmids, baculovirus vectors, a phagemid, a phage derivative, an animal virus, and a cosmid. In some embodiments, at least one nucleic acid sequence encoding a variant SGSH is encoded by a nucleic acid sequence in a gene therapy vector. In some embodiments, the gene therapy vector encodes at least one of the amino acid sequences in Table 1 (SEQ ID NOs: 2-20). In some embodiments, the gene therapy vector is an adeno-associated virus (AAV), an adenoviral vector, a lentiviral vector, adeno-associated virus (AAV), a retrovirus, a lentivirus, a herpes simplex virus.

### Signal Sequences

The variant SGSH protein may further comprise a signal peptide. In some variant SGSH proteins, the signal peptide is the native human SGSH signal peptide (SEQ ID NO:21). In other variant SGSH proteins, the signal peptide is a non-native signal peptide, such as a human immunoglobulin heavy chain binding protein (BiP) or a Gaussia signal peptide. The BiP peptide may be the native human BiP peptide or a modified BiP sequence, such as the modified BiP sequences in Table 2 and those described in WO 2012/071422 and U.S. Pat. No. 9,279,007. In some embodiments, a signal peptide comprises SEQ ID NO: 22, 23, 24, 25, 26 or 27.

TABLE 2
Signal Peptide Sequences

| | | SEQ |
|---|---|---|
| Signal Peptide | Amino Acid Sequence | ID NO: |
| Native human | MSCPVPACCALLLVLGLCRA | 21 |
| SGSH | | |
| Native human | MKLSLVAAMLLLLSAARA | 22 |
| BiP | | |
| Modified BiP-1 | MKLSLVAAMLLLLSLVAAMLLLLSAARA | 23 |
| Modified BiP-2 | MKLSLVAAMLLLLWVALLLLSAARA | 24 |
| Modified BiP-3 | MKLSLVAAMLLLLSLVALLLLSAARA | 25 |
| Modified BiP-4 | MKLSLVAAMLLLLALVALLLLSAARA | 26 |
| Gaussia | MGVKVLFALICIAVAEA | 27 |

### Variant IGF2 Sequences

In some embodiments, the variant SGSH protein further comprises a component that enhances cellular uptake and delivery to lysosomes by binding to the cation-independent mannose phosphate receptor (CIMPR). In some embodiments, the variant SGSH further comprises a variant human IGF2 peptide (vIGF2). In some embodiments, the vIGF2 peptide comprises the amino acid sequence of any one of SEQ ID NOs: 28-91 (see Table 3). In some embodiments, the vIGF2 peptide comprises an amino acid sequence at least about 70%, about 75%, about 80%, about 85%, about 90%, about 95%, about 96%, about 97%, or about 98% identical to the amino acid sequence of any one of SEQ ID NO: 28-91. In some embodiments, the vIGF2 peptide comprises an amino acid sequence that is at least 98% identical to SEQ ID NO:40 or SEQ ID NO:41. In some embodiments, the vIGF2 peptide comprises the amino acid sequence of SEQ ID NO:40 or SEQ ID NO: 41.

TABLE 3
IGF2 Amino Acid Sequences (variant residues are underlined)

| Peptide | Sequence | SEQ ID NO |
|---|---|---|
| Wildtype | AYRPSETLCGGELVDTLQFVCGDRGFYFSRPASRVSRRS | 28 |
| | RGIVEECCFRSCDLALLETYCATPAKSE | |
| F26S | AYRPSETLCGGELVDTLQFVCGDRGSYFSRPASRVSRRS | 29 |
| | RGIVEECCFRSCDLALLETYCATPAKSE | |
| Y27L | AYRPSETLCGGELVDTLQFVCGDRGFLFSRPASRVSRRS | 30 |
| | RGIVEECCFRSCDLALLETYCATPAKSE | |
| V43L | AYRPSETLCGGELVDTLQFVCGDRGFYFSRPASRVSRRS | 31 |
| | RGILEECCFRSCDLALLETYCATPAKSE | |
| F48T | AYRPSETLCGGELVDTLQFVCGDRGFYFSRPASRVSRRS | 32 |
| | RGIVEECCTRSCDLALLETYCATPAKSE | |
| R49S | AYRPSETLCGGELVDTLQFVCGDRGFYFSRPASRVSRRS | 33 |
| | RGIVEECCFSSCDLALLETYCATPAKSE | |
| S50I | AYRPSETLCGGELVDTLQFVCGDRGFYFSRPASRVSRRS | 34 |
| | RGIVEECCFRICDLALLETYCATPAKSE | |
| A54R | AYRPSETLCGGELVDTLQFVCGDRGFYFSRPASRVSRRS | 35 |
| | RGIVEECCFRSCDLRLLETYCATPAKSE | |
| L55R | AYRPSETLCGGELVDTLQFVCGDRGFYFSRPASRVSRRS | 36 |
| | RGIVEECCFRSCDLARLETYCATPAKSE | |
| F26S, Y27L, | AYRPSETLCGGELVDTLQFVCGDRGSLFSRPASRVSRRS | 37 |
| V43L, F48T, | RGILEECCTSICDLRRLETYCATPAKSE | |
| R49S, S50I, | | |
| A54R, L55R | | |
| Δ1-6, Y27L, | TLCGGELVDTLQFVCGDRGFLFSRPASRVSRRSRGIVEE | 38 |
| K65R | CCFRSCDLALLETYCATPARSE | |
| Δ1-7, Y27L, | LCGGELVDTLQFVCGDRGFLFSRPASRVSRRSRGIVEEC | 39 |
| K65R | CFRSCDLALLETYCATPARSE | |
| Δ1-4, E6R, | SRTLCGGELVDTLQFVCGDRGFLFSRPASRVSRRSRGIV | 40 |
| Y27L, K65R | EECCFRSCDLALLETYCATPARSE | |
| Δ1-4, E6R, | SRTLCGGELVDTLQFVCGDRGFLFSRPASRVSRRSRGIV | 41 |
| Y27L | EECCFRSCDLALLETYCATPAKSE | |
| E6R | AYRPSRTLCGGELVDTLQFVCGDRGFYFSRPASRVSRRS | 42 |
| | RGIVEECCFRSCDLALLETYCATPAKSE | |
| Δ1-4, E6R, | SRTLCGGELVDTLQFVCGDRGFLFSRPASRVSRRSRGIV | 43 |
| Y27L, S50E, | EECCFRECDLALLETYCATPARSE | |
| K65R | | |
| Cleavable IGF2 | GGGGSGGGGSRPRAVPTQ | 44 |
| variant-N | | |
| terminal | | |
| Cleavable IGF2 | YIPAKQGLQGAQMGQPGGGGGGGG | 45 |
| variant-C | | |
| terminal | | |
| Cleavable IGF2 | RPRAVPTQGGSGSGSTSS | 46 |
| variant-C | | |
| terminal | | |
| Cleavable IGF2 | SRTLCGGELVDTLQFVCGDRGFLFSRPASRVSRRSRGIV | 47 |
| variant-N | EECCFRSCDLALLETYCATPARSEGGGGSGGGGSRPRAV | |
| terminal | PTQ | |
| Cleavable IGF2 | YIPAKQGLQGAQMGQPGGGGSGGGGSRTLCGGELVDTLQ | 48 |
| variant-C | FVCGDRGFLFSRPASRVSRRSRGIVEECCFRSCDLALLE | |
| terminal | TYCATPARSE | |
| Cleavable IGF2 | RPRAVPTQGGSGSGSTSSSRTLCGGELVDTLQFVCGDRG | 49 |
| variant-C | FLFSRPASRVSRRSRGIVEECCFRECDLALLETYCATPA | |
| terminal | RSE | |
| vIGF2-1 | SRTLCGGELVDTNQFVCGDRGFLFSRGASRVSRGSRGIV | 50 |
| (vIGF2_1_NGG | EWCCFRGCDLALLETYCATPMRGE | |
| WGMG) | | |
| vIGF2-2 | SRTLCGGELVDTLQFVCGDRGFLFSRGASRVSRGSRGIV | 51 |
| (vIGF2_2_GG | EWCCFRGCDLALLETYCATPMRGE | |
| WGMG) | | |
| vIGF2-3 | SRTLCGGELVDTNQFVCGDRGFLFSRGASRVSRGSRGIV | 52 |
| (vIGF2_3_NGG | EECCFRGCDLALLETYCATPMRGE | |
| GMG) | | |
| vIGF2-4 | SRTLCGGELVDTLQFVCGDRGFLFSRPIVEECCFRSCDL | 53 |
| (vIGF2_4_Δ32- | ALLETYCATPARSE | |
| 41, 53aa) | | |
| vIGF2-5 (vIGF2 | SRTLCGGELVDTLQFVCGDRGFLFSRGIVEECCFRSCDL | 54 |
| Δ 30-39, 53aa) | ALLETYCATPARSE | |
| vIGF2-6 (vIGF2 | SRTLCGGELVDTLQFVCGDRGFLFSRPAGIVEECCFRSC | 55 |
| Δ 33-40, 55aa) | DLALLETYCATPARSE | |
| vIGF2-7 (vIGF2 | SRTLCGGELDDTLQFVCGDRGALRSRGIDEECCFRSCDL | 56 |
| Δ 30- | ALLETYCATPARSE | |
| 39/V14D/F28R/ | | |
| V43D/F26A) | | |
| vIGF2-8 | SRTLCGGELVDTLQFVCGRRGELFSRPASRVSRRSRGIV | 57 |
| (vIGF2_8_REE) | EECCFRECDLALLETYC ATPARSE | |
| vIGF2-9 | SRTLCGGELVDTLQFVCGRRGELFSRPAGIVEECCFREC | 58 |
| (vIGF2_9_Δ | DLALLETYCATPARSE | |
| 30-39-REE; | | |
| vIGF2) | | |
| vIGF2-10 | SRTLCGGELVDTLQFVCGRRGFLFSRPASRVSRRSRGIV | 59 |
| (vIGF2_1Q; | EECCFRSCDLALLETYCATPARSE | |
| vIGF2 D23R) | | |
| vIGF2-11 | SRTLCGGELVDTLQWVCGDRGFLFSRPASRVSRRSRGIV | 60 |
| (vIGF2_2Q; | EECCFRSCDLALLETYCATPARSE | |
| vIGF2 F19W) | | |
| vIGF2-12 | SRTLCGGELVDWLQFVCGDRGFLFSRPASRVSRRSRGIV | 61 |
| (vIGF2_3Q; | EECCFRSCDLALLETYCATPARSE | |
| vIGF2 T16W) | | |
| vIGF2-13 | SRTLCGGELVDTLQFVCGKRGFLFSRPASRVSRRSRGIV | 62 |
| (vIGF2_4Q; | EECCFRSCDLALLETYCATPARSE | |
| vIGF2 D23K) | | |
| vIGF2-14 | SRTLCGGELVDYLQFVCGDRGFLFSRPASRVSRRSRGIV | 63 |
| (vIGF2_5Q; | EECCFRSCDLALLETYCATPARSE | |
| vIGF2 T16Y) | | |
| vIGF2-15 | SRTLCGGELVDTLQFVCGDRGELFSRPASRVSRRSRGIV | 64 |
| (vIGF2_6Q; | EECCFRSCDLALLETYCATPARSE | |
| vIGF2 F26E) | | |
| vIGF2-16 | SRTLCGGELVDVLQFVCGDRGFLFSRPASRVSRRSRGIV | 65 |
| (vIGF2_7Q; | EECCFRSCDLALLETYCATPARSE | |
| vIGF2 T16V) | | |
| vIGF2-17 | SRTLCGGELVDTLQFVCGDRGELFSRPASRVSRRSRGIV | 66 |
| (vIGF2_8Q; | EECCFRECDLALLETYCATPARSE | |
| vIGF2 S50E) | | |
| vIGF2-18 | SRTLCGGELVDTLQFVCGDRGFLFSRPASRVSRRSRGIV | 67 |
| (vIGF2_9Q; | EECCFRDCDLALLETYCATPARSE | |
| vIGF2 S50D) | | |
| vIGF2-19 | SRTLCGGELVDTLQFVCGDRGSLFSRPASRVSRRSRGIL | 68 |
| (vIGF2 F26S | EECCFRSCDLALLETYCATPARSE | |
| V43L) | | |
| vIGF2-20 | SRTLCGGELVDTLQFVCGDRGFLFSRPASRVSRRSRGIL | 69 |
| (vIGF2 V43L) | EECCFRSCDLALLETYCATPARSE | |
| vIGF2-21 | SRTLCGGELEDTLQFVCGDRGSLRSRPASRVSRRSRGIE | 70 |
| (vIGF2_ESRE; | EECCFRSCDLALLETYCATPARSE | |
| vIGF2 V14E | | |
| F26S F28R | | |
| V43E) | | |
| vIGF2-22 | SRTLCGGELVDTLQFVCGDRGFLFSRGGGGSRGIVEECC | 71 |
| (vIGF2 Δ 31- | FRSCDLALLETYCATPARSE | |
| 38GGGG) | | |
| vIGF2-23 | SRTLCGGELVDTLQFVCGDRGFLFSGGGSGIVEECCFRS | 72 |
| (vIGF2 Δ 30- | CDLALLETYCATPARSE | |
| 40GGGG) | | |
| vIGF2-24 | SRTLCGGELVDTLQFVCGDRGFLFSRGGGGSRGILEECC | 73 |
| (vIGF2 Δ 31- | FRSCDLALLETYCATPARSE | |
| 38GGGG | | |
| V43L) | | |
| vIGF2-25 | SRTACGGELVDTLQFVCGDRGFLFSRPASRVSRRSRGIV | 74 |
| (vIGF2 L8A) | EECCFRSCDLALLETYCATPARSE | |
| vIGF2-26 | SQAACGGELVDTLQFVCGDRGFLFSRPASRVSRRSRGIV | 75 |
| (vIGF2 R6Q | EECCFRSCDLALLETYCATPARSE | |
| T7A L8A) | | |
| vIGF2-27 | SRTLCGGELVDTLQFVCGDEGFLFSRPASEVSEESRGIV | 76 |
| (vIGF2 R24E | EECCFRSCDLALLETYCATPARSE | |
| R34ER37E | | |
| R38E) | | |
| vIGF2-28 | SRTLCGGELVDTLQFVCGDEGFLFSRPASEVSRRSRGIV | 77 |
| (vIGF2 R24E | EECCFRSCDLALLETYCATPARSE | |
| R34E) | | |
| vIGF2-29 | SRTLCGGELVDTLQFVCGRRGFLFSRPASRVSRRSRGIV | 78 |
| (vIGF2 D23R | | |
| S40D) | EECCFRDCDLALLETYCATPARSE | |
| vIGF2-30 | SRTLCGGELVDVLQFVCGRRGFLFSRPASRVSRRSRGIV | 79 |
| (vIGF2 T16V | EECCFRDCDLALLETYCATPARSE | |
| D23R S50D) | | |
| vIGF2-31 | SRTLCGGELVDTLQFVCGDRGFLFSRGGGGSRGILEECC | 80 |
| (vIGF2 Δ 31- | FRDCDLALLETYCATPARSE | |
| 38GGGG V43L | | |
| S50D) | | |
| vIGF2-32 | SRTLCGGELVDTLQFVCGDRGFLFSRGGGGSRGILEECC | 81 |
| (vIGF2 Δ 31- | FRECDLALLETYCATPARSE | |
| 38GGGG V43L | | |
| S50E) | | |
| vIGF2-33 | SRTLCGGELVDTLQFVCGDRGFLFRLPSRPVSRHSHRRS | 82 |
| (vIGF2-N1) | RGIVEECCFQRCNLALLETYCATPARSE | |
| vIGF2-34 | SRTLCGGELVDTLQFVCGDRGFLERLPSRPVSRHSHRRS | 83 |
| (vIGF2-N1 | RGILEECC FQECNLALLETYCATPARSE | |
| V43L S50E) | | |
| vIGF2-1 R38G | SRTLCGGELVDTLQFVCGDRGFLFSRPASRVSRGSRGIV | 84 |
| | EECCFRSCDLALLETYCATPARSE | |
| vIGF2-2 R38G, | SRTLCGGELVDTLQFVCGDRGFLFSRPASRVSRGSRGIV | 85 |
| E45W | EWCCFRSCDLALLETYCATPARSE | |
| vIGF2-3 R38G, | SRTLCGGELVDTLQFVCGDRGFLFSRPASRVSRGSRGIV | 86 |
| E45W, S50G | EWCCFRGCDLALLETYCATPARSE | |
| vIGF2-4 P31G, | SRTLCGGELVDTLQFVCGDRGFLFSRGASRVSRGSRGIV | 87 |
| R38G, E45W, | EWCCFRGCDLALLETYCATPARSE | |
| S50G | | |
| vIGF2-5 L17N, | SRTLCGGELVDTNQFVCGDRGFLFSRGASRVSRGSRGIV | 88 |
| P31G, R38G, | EWCCFRGCDLALLETYCATPARSE | |
| E45W, S50G | | |
| vIGF2-6 L17N, | SRTLCGGELVDTNQFVCGDRGFLFSRGASRVSRGSRGIV | 89 |
| P31G, R38G, | EWCCFRGCDLALLETYCATPARGE | |
| E45W, S50G, | | |
| S66G | | |
| vIGF2-7 L17N, | SRTLCGGELVDTNQFVCGDRGFLFSRGASRVSRGSRGIV | 90 |
| P31G, R38G, | EWCCFRGCDLALLETYCATPMRGE | |
| E45W, S50G, | | |
| A64M, S66G | | |
| vIGF2-8 S5L, | LRTLCGGELVDTNQFVCGDRGFLFSRGASRVSRGSRGIV | 91 |
| L17N, P31G, | EWCCFRGCDLALLETYCATPMRGE | |
| R38G, E45W, | | |
| S50G, A64M, | | |
| S66G | | |

### Therapeutic Variant Human SGSH Proteins for Enzyme Replacement Therapy

Therapeutic variant human SGSH proteins produced for enzyme replacement therapy are provided herein. The provided variant human SGSH proteins are, in some embodiments, engineered to improve delivery of the therapeutic protein. For example, in some instances fusion protein may not achieve the intended treatment if an insufficient amount of the therapeutic fusion protein is delivered into the cells in need of the therapeutic protein, due to, for example, physical and/or biological barriers that impede distribution of the therapeutic protein to the site where needed. Even if the therapeutic protein is transported out of the vasculature to the interstitial space within the tissue (e.g., muscle fibers), adequate therapeutic effects are not assured. For effective treatment of lysosomal storage disorders, a therapeutically effective amount of the therapeutic protein must undergo cellular endocytosis and lysosomal delivery to result in a meaningful efficacy. The present disclosure addresses these issues by providing fusion proteins comprising a peptide that enables endocytosis of the therapeutic protein into a target cell for treatment resulting in efficacious treatment. In some embodiments, the peptide that enables endocytosis is a peptide that binds the CIMPR. In some embodiments, the peptide that binds the CIMPR is a vIGF2 peptide. Such variant human SGSH proteins have increased delivery into or cellular uptake by cells needing such proteins and target the therapeutic protein to a subcellular location (e.g., a lysosome). In some embodiments, the peptide is an IGF2 peptide or variant thereof, which can target a therapeutic protein to the lysosome. Therapeutic proteins for enzyme replacement therapy or gene therapy comprising a vIGF2 peptide are provided herein. Exemplary proteins are provided in Table 1 above.

### Gene Therapy Vectors and Compositions

In some embodiments, the nucleic acid encoding the SGSH polypeptide or provided herein is cloned into a vector including, but not limited to a plasmid, a phagemid, a phage derivative, an animal virus, a bacterial artificial chromosome, or a cosmid. Vectors of particular interest include expression vectors, replication vectors, probe generation vectors, gene therapy vectors and sequencing vectors.

Further, the vector encoding the encoding the SGSH polypeptide provided herein, in some embodiments, is provided to a cell in the form of a viral vector. Viral vector technology is described, e.g., in Sambrook et al., 2012, Molecular Cloning: A Laboratory Manual, volumes 1-4, Cold Spring Harbor Press, NY), and in other virology and molecular biology manuals. Viruses that are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

Also provided herein are compositions and systems for gene transfer. A number of virally based systems have been developed for gene transfer into mammalian cells. For example, retroviruses provide a convenient platform for gene delivery systems. A selected gene, in some embodiments, is inserted into a vector and packaged in retroviral particles using suitable techniques. The recombinant virus is then isolated and delivered to cells of the subject either in vivo or ex vivo. A number of retroviral systems are suitable for gene therapy. In some embodiments, adenovirus vectors are used. A number of adenovirus vectors are suitable for gene therapy. In some embodiments, adeno-associated virus vectors are used. A number of adeno-associated viruses are suitable for gene therapy. In some embodiments, lentivirus vectors are used.

Gene therapy constructs provided herein comprise a vector (or gene therapy expression vector) into which the gene of interest (e.g., a gene encoding a variant SGSH protein) is cloned or otherwise which includes the gene of interest in a manner such that the nucleotide sequences of the vector allow for the expression (constitutive or otherwise regulated in some manner) of the gene of interest. The vector constructs provided herein include any suitable gene expression vector that is capable of being delivered to a tissue of interest and that will provide for the expression of the gene of interest in the selected tissue of interest.

In some embodiments, the vector is an adeno-associated virus (AAV) vector. AAV vectors are DNA parvoviruses that are nonpathogenic for mammals. Briefly, AAV-based vectors have the rep and cap viral genes that account for 96% of the viral genome removed, leaving the two flanking AAV inverted terminal repeats (ITRs) which are used to initiate viral DNA replication and packaging. In some embodiments, an AAV ITR is an AAV2 ITR. Certain AAV vectors are useful for gene therapy, in part, because of their capacity to cross the blood-brain barrier and transduce neuronal tissue.

In some embodiments, an AAV vector provided herein is self-complementary. As used herein, the term “self-complementary” when referring to an AAV vector refers to an AAV vector comprising a nucleic acid (i.e., a DNA) that forms a dimeric inverted repeat molecule that spontaneously anneals, resulting in earlier and more robust transgene expression compared with conventional single-strand (ss) AAV genomes. See, e.g., McCarty, Molecular Therapy 16 (10): 1648-1656 (2008). Unlike conventional ssAAV, self-complementary AAV (scAAV) can bypass second-strand synthesis, the rate-limiting step for gene expression. Moreover, double-stranded scAAV is less prone to DNA degradation after viral transduction, thereby increasing the number of copies of stable episomes. In some embodiments, an AAV vector provided herein is single-stranded.

In methods and compositions provided herein, AAV of any serotype may be used. In any of the methods and compositions provided herein, an AAV vector may be an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAV-PHP.B vector, an AAVhu68 vector, an AAV-DJ vector, an AAV2/9 vector, a TM-AAV6 vector, an AAV-PHP.A vector, an AAV-PHP. S vector, or an AAV-PHPeB vector, or another AAV vector suitable for gene therapy.

An example of a promoter that is capable of expressing a variant SGSH transgene in a mammalian T-cell, such as an EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving expression from transgenes cloned into a lentiviral vector (see, e.g., Milone et al., Mol. Ther. 17 (8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences are sometimes also used, including, but not limited to the chicken β actin promoter, the P546 promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, gene therapy vectors are not contemplated to be limited to the use of constitutive promoters. Inducible promoters are also contemplated here. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline-regulated promoter.

In some embodiments, a promoter is the chicken beta-actin (CBA) promoter, the GUSB240 promoter, the GUSB379 promoter, the HSVTK promoter, the CMV promoter, the CB7 promoter, the SV40 early promoter, the SV40 late promoter, the metallothionein promoter, the murine mammary tumor virus (MMTV) promoter, the Rous sarcoma virus (RSV) promoter, the polyhedrin promoter, the EF-1 alpha promoter, the dihydrofolate reductase (DHFR) promoter or the phosphoglycerol kinase (PGK) promoter.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements is often increased to 50 bp apart before activity begins to decline. Depending on the promoter, it appears that individual elements function cither cooperatively or independently to activate transcription.

In some embodiments, a vector provided for expressing a variant SGSH protein further comprises or encodes a woodchuck hepatitis virus post-transcriptional element (WPRE). See, e.g., Wang and Verma, Proc. Natl. Acad. Sci., USA, 96:3906-3910 (1999). In some embodiments, a vector comprises or encodes a hepatitis B virus posttranscriptional regulatory element (HBVPRE) and/or a RNA transport element (RTE). In some embodiments, the WPRE or HBVPRE sequence is any of the WPRE or HBVPRE sequences disclosed in U.S. Pat. No. 6,136,597 or U.S. Pat. No. 6,287,814.

In some embodiments, a vector provided for expressing a variant SGSH protein further comprises or encodes a polyadenylation (polyA) signal sequence. As used herein, a “polyadenylation signal sequence” refers to a DNA sequence that when transcribed regulates the addition of a polyA tail to the mRNA transcript. In some embodiments, a poly A signal sequence is a SV40, human, bovine or rabbit polyA signal sequence. In some embodiments, a polyA signal sequence is a SV40 polyA signal sequence. In some embodiments, a polyA signal sequence is a β-globin polyA signal sequence. In some embodiments, a polyA signal sequence is a human growth hormone polyA signal sequence or a bovine growth hormone polyA signal sequence.

In some embodiments, a vector provided for expressing a variant SGSH protein further comprises or encodes a Kozak sequence (for example, a DNA sequence transcribed to an RNA Kozak sequence). In some embodiments, a vector comprises a Kozak sequence upstream of the transgene. In some embodiments, the Kozak sequence is encoded by GCCACC (SEQ ID NO: 92). In some embodiments, the Kozak sequence (e.g., RNA Kozak sequence) comprises or consists of ACCAUGG (SEQ ID NO: 93), GCCGCCACCAUGG (SEQ ID NO: 94), CCACCAUG (SEQ ID NO: 95) or CCACCAUGG (SEQ ID NO: 96).

In some embodiments, a vector provided herein for expressing a variant SGSH protein further comprises a TATA transcriptional regulatory activation site (see, e.g., Francois et al., (2005) J. Virol. 79 (17): 11082-11094).

In order to assess the expression of a vector encoding the variant SGSH polypeptides provided herein, the expression vector can be introduced into a cell having a selectable marker gene or a reporter gene or both to facilitate identification and selection of transfected, infected or transduced cells. In other aspects, the selectable marker is often carried on a separate piece of DNA and used in a co-transfection procedure. Both selectable markers and reporter genes are sometimes flanked with appropriate regulatory sequences to enable expression in the host cells. Useful selectable markers include, for example, antibiotic-resistance genes.

Methods and compositions for introducing and expressing genes into a cell are suitable for methods herein. In the context of an expression vector, the vector is readily introduced into a host cell, e.g., mammalian, bacterial, yeast, or insect cell by any method in the art, such as calcium phosphate precipitation, lipofection, particle bombardment, microinjection, gene gun, electroporation (see, e.g., Sambrook et al., 2012, Molecular Cloning: A Laboratory Manual, volumes 1-4, Cold Spring Harbor Press, NY).

Other means and compositions for introducing a polynucleotide encoding a variant SGSH into a host cell include colloidal dispersion systems, such as macromolecule complexes, nanocapsules, microspheres, beads, targeted nanoparticles, and lipid-based systems including oil-in-water emulsions, micelles, mixed micelles, nucleic acid-lipid particles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/nucleic acid (such as lipid/DNA or lipid/mRNA) or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.; in some embodiments, dicetyl phosphate (“DCP”) is obtained from K & K Laboratories (Plainview, N.Y.); cholesterol (“Choi”), in some embodiments, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids, in some embodiments, assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to the encoding the variant SGSH polypeptide provided herein, in order to confirm the presence of the recombinant DNA sequence in the host cell, a variety of assays are contemplated to be performed. Such assays include, for example, “molecular biological” assays suitable for methods herein, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope herein.

The present disclosure further provides a vector comprising a variant SGSH polypeptide provided herein, encoding nucleic acid molecule. In one aspect, a therapeutic fusion protein vector is capable of being directly transduced into a cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the variant SGSH polypeptide provided herein in mammalian cells. In one aspect, the mammalian cell is a human cell.

### Pharmaceutical Compositions

In additional aspects, there are provided pharmaceutical compositions comprising a therapeutically effective amount a variant SGSH polypeptide or a nucleic acid construct encoding any of the variant SGSH polypeptides provided herein, or a gene therapy vector for delivery of a transgene encoding any of the variant SGSH polypeptides provided here, along with a pharmaceutically acceptable carrier or excipient. In some embodiments, the excipient comprises a non-ionic, low-osmolar compound, a buffer, a polymer, a salt, or a combination thereof.

In additional aspects, there are provided pharmaceutical compositions comprising any one of the gene therapy vectors provided herein and a pharmaceutically acceptable carrier or excipient for use in treating a genetic disorder. In further aspects, there are provided pharmaceutical composition comprising any one of the nucleic acid constructs provided herein and a pharmaceutically acceptable carrier or excipient for use in preparation of a medicament for treatment of a genetic disorder. In some embodiments, the genetic disorder is a lysosomal storage disorder. In some embodiments, the genetic disorder is MPS IIIA.

In some embodiments, the composition is formulated for administration intrathecally, intraocularly, intravitreally, retinally, subretinally, intravenously, intramuscularly, subcutaneously, intraventricularly, intracerebrally, intracerebellarly, intracisternally, intracerebroventrically, intraparenchymally, surgically, intradermally or topically.

### Methods of Treatment

Provided herein are methods of treating MPS IIIA comprising administering to a subject in need thereof a therapeutically effective amount of at least one of the variant SGSH proteins provided herein. In some embodiments, the variant SGSH is administered as an enzyme replacement therapy. In some embodiments, the variant SGSH protein comprises the amino acid sequence of any one of SEQ ID NO:2-20. In some aspects, the variant SGSH protein comprises the amino acid sequence of SEQ ID NO:2, SEQ ID NO:3 or SEQ ID NO:4.

Further provided herein are gene therapy methods of treating MPS IIIA by replacing or supplementing a defective SGSH gene in a patient in need thereof. In some embodiments, the gene therapy method comprises administering to a subject in need thereof a therapeutically effective amount of a pharmaceutical formulation comprising a nucleic acid encoding at least one of the variant SGSH polypeptide provided herein. In some embodiments, the gene therapy is delivered using a viral vector, such as an AAV. In some embodiments, a viral vector comprises a nucleic acid encoding a protein comprising the amino acid sequence of any one of SEQ ID NO:2-20.

In some embodiments, a therapeutic gene encoding a variant SGSH is delivered using a viral vector. In some embodiments, the viral vector is an AAV or an AAV derivative. In some embodiments, the AAV is an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAV-PHP.B vector, an AAVhu68 vector, an AAV-DJ vector, an AAV2/9 vector, a TM-AAV6 vector, an AAV-PHP.A vector, an AAV-PHP.S vector, or an AAV-PHPeB vector.

In one or more embodiments, the pharmaceutical formulation is administered surgically, intrathecally, intravenously, intramuscularly, intracisternally, intracerebroventrically, subcutaneously, subretinally, intravitreally, intraparenchymally, intradermally or topically.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular systems biology 7:539. For the purposes of calculating identity to the sequence, extensions, such as tags, are not included.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: In Silico Analysis of SGSH Variants

SGSH was engineered for better dimer stability as well as for better cross correction. For better cross correction, the native signal sequence was replaced with BiP signal sequence, and the vIGF2 tag was added to the C terminus of SGSH, with a proteolytic cleavage site and a GS linker in between the two.

SGSH is a functional homodimer. In order to increase the stability of the SGSH dimer, Cyrus Bench was used to select and mutate all residues located at the dimer interface. Cyrus Bench allows the user to make mutations, and then calculates the resulting Rosetta energy score of the overall structure resulting from the mutations. A Monte Carlo method of sampling conformational changes is used to generate each structure, and thus it is best practice to calculate an energy score several times for each mutant generated, in order to have statistical significance in energy scores between different mutant structures.

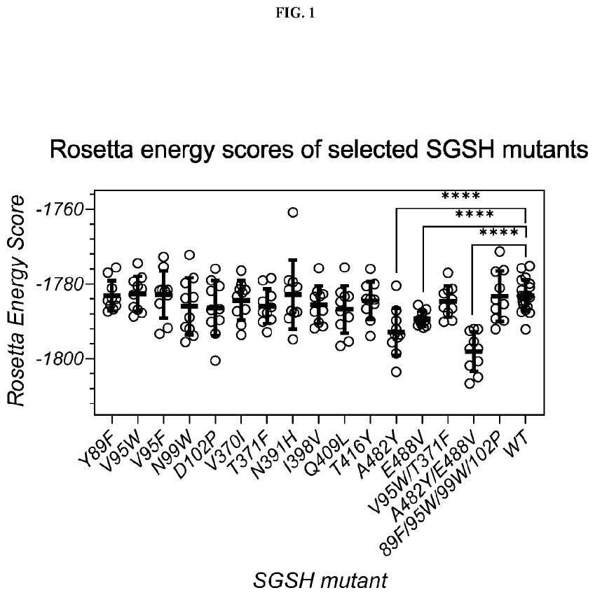

Hundreds of mutations and combinations of mutations were sampled in silico, some examples of which are shown in FIG. 1. Three mutants were found to have a consistent and significant reduction in their Rosetta energy score, as compared to WT. These were SGSH A482Y, SGSH E488V, and SGSH A482Y E488V.

### Example 2: In Vitro Testing of SGSH Variants

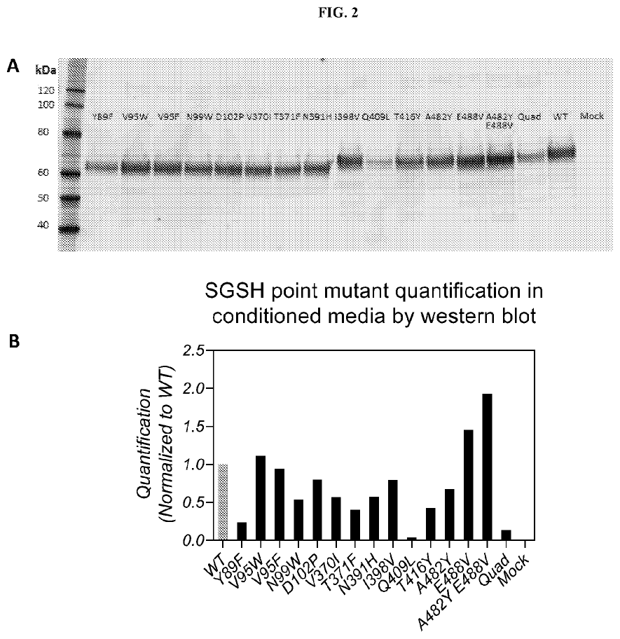

Several mutants were generated for in vitro testing of expression and secretion, including the most promising three mutants. All of the constructs were made to contain the BiP signal sequence instead of the endogenous signal sequence, including the WT construct. In this case, WT refers to the unaltered sequence of the enzyme after the removal of the signal sequence. Each construct was cloned into a mammalian expression vector, and then used to transfect Freestyle 293F cells, using polyethylenimine (PEI) transfection reagent. Valproic acid was added 24 hours post-transfection, and cells and conditioned media were harvested four days post-transfection. Western blots were run on the conditioned media, in order to show the effect of the mutations on the amount of SGSH secreted into the media. An anti-SGSH monoclonal antibody from Abcam (ab200346) was used for the blots. A representative blot is shown in FIG. 2a, and the quantification of the blot is shown in FIG. 2b. Single mutant E488V and double mutant A482Y E488V are found in the media at levels 1.5 times higher, and 2 times higher that WT, respectively. Both of these constructs were predicted in silico to have a lower energy score, and therefore better stability. The A482Y single mutant was also predicted to have a lower energy score, but did not outperform WT expression/secretion in vitro. Other mutations, such as Y89F and Q409L expressed much more poorly than WT, despite their in silico energy scores being on par with that of WT.

A fluorimetric enzyme activity assay was performed on the conditioned media collected from the transfections with the different mutants. The purpose was to show that the mutations (particularly the ones yielding higher expression/secretion) did not impede the enzymatic activity of the SGSH. This activity assay is based on the one published by Karpova, et al. (Karpova, et al. 1996. J Inherit Metab Dis). This is a coupled enzyme assay that uses the fluorogenic substrate 4-Methylumbelliferyl 2-Sulfamino-2-deoxy-α-D-glucopyranoside (4-MU-GlcNS), which is acted upon by SGSH. The product of the reaction is a substrate for the enzyme α-glucosidase, which is used in the assay as the second half of the coupled reaction and produces 4-MU, which fluoresces and can be quantified to measure SGSH activity. Published protocols perform this assay as a two-step reaction in two separate buffers at different pH, as the substrate is not ideal for either enzyme in the coupled reaction, and the reaction with yeast α-glucosidase is slower than the reaction performed by SGSH. Our amended method developed in-house uses much larger quantities of human lysosomal α-glucosidase, which has a pH optimum closer to that of SGSH. In this way, both reactions can be performed together, and the overabundance of α-glucosidase counteracts the slower reaction time for the enzyme. This takes the published assay time of over 40 hours down to six hours.

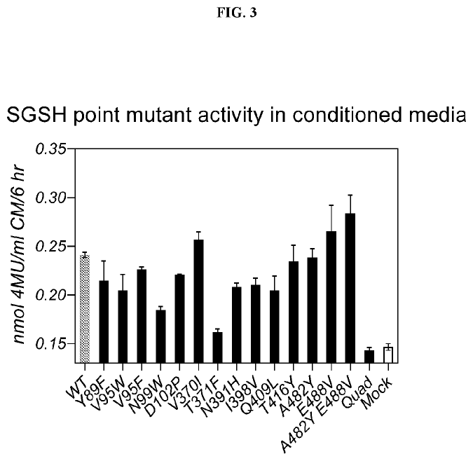

The fluorimetric activity assay showed that the constructs of interest, specifically those containing the mutations A482Y and/or E488V, retained their enzymatic activity (FIG. 3).

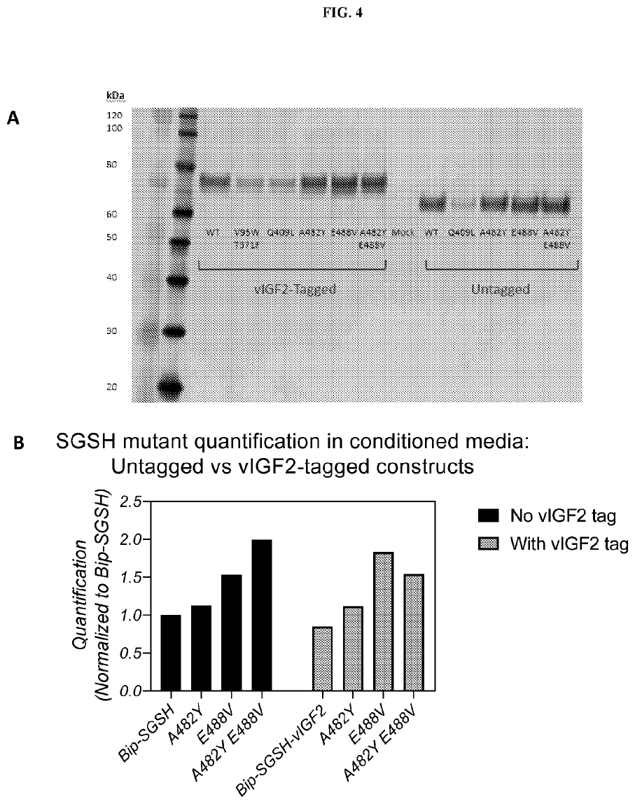

To improve the potential for cross-correction between adjacent cells, a fusion protein was designed to include the vIGF2 tag at the C-terminus of the most promising SGSH mutants, linked by a proteolytic cleavage site and a GS linker. The tag was placed at the C-terminus rather than at the N-terminus of each construct because the structure of SGSH shows that the C-termini of both monomers are unobstructed and easily accessible. In addition, prior testing of SGSH expression with N-terminal versus C-terminal tag showed better expression when the vIGF2 tag was at the C-terminus. Expression/secretion of the tagged SGSH mutants of interests is shown in FIG. 4. The constructs were expressed and harvested in the same manner as described above for the untagged mutants. FIG. 4 shows that the expression of the mutants of interest, specifically the E488V single mutant and the A482Y/E488V double mutant, is not substantially affected by tagging with vIGF2. Both tagged mutants express at levels greater than 50% higher than the untagged WT. The double mutant was chosen as the lead engineered construct going forward, for additional in vitro assays as well as for in vivo work.

A second activity assay was also developed, based on the activity assay published by Yi, et al. (Yi, et al. 2018. Mol Genet Metab). This is the assay that was used to assess CIMPR binding (see below) as well as SGSH activity in animal tissues. This is a one-step activity assay that directly measures the product formed when SGSH acts upon the substrate 2-sulfamate-2-deoxy-1-α-(2-naphthyl)-glucopyranoside via GC/MS/MS. The enzymatic reaction time for the assay can be as little as six hours. The published method measures SGSH activity in dried blood spots, but was further developed internally to work with several animal tissue matrices. This activity assay is an improvement over the fluorescence-based activity assay because it removes the need for a coupled reaction, instead measuring directly the activity of SGSH, and also allows for more precise measurements with MS.

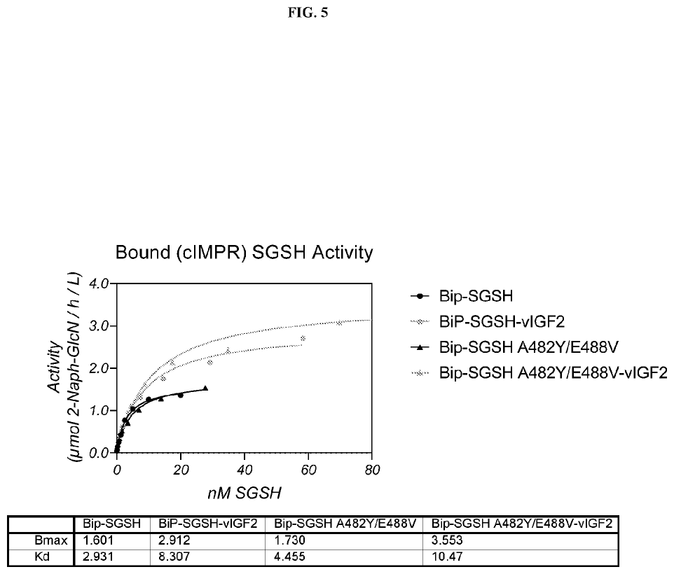

Conditioned media from the expression of the constructs described above were used to test their affinity for the cation-independent mannose-6-phosphate receptor (CIMPR). This assay acts as a surrogate to measure how well the tagged and untagged proteins are taken up into lysosomes. In the CIMPR binding assay, a 96-well plate was coated with receptor, washed, and blocked with BSA. Conditioned media was serially diluted to give a series of decreasing concentrations and incubated in the receptor-coated wells. After incubation the plate was washed to remove any unbound enzyme. Bound SGSH was measured by performing the Liquid Chromatography Tandem Mass Spectrometry (LC-MS/MS)-based activity assay in the wells containing the bound enzyme. The measured activity was plotted against the initial amount of SGSH that was incubated in the wells of the plate (FIG. 5). This initial SGSH concentration in the conditioned media was calculated by measuring SGSH activity in the conditioned media against a standard curve made from commercial SGSH purchased from Origene (cat. #TP720741). The binding curves show that for each concentration tested, more of the vIGF2-tagged SGSH enzyme than the untagged SGSH enzyme binds to the CIMPR-coated plates. This applies for both the mutant and the unmodified SGSH enzyme sequence.

### Example 3: In Vivo Mouse Efficacy Study

The mice used in the study were homozygous SGSH knockouts and WT controls. Two doses of virus were tested for each SGSH construct, via ICV administration. Ten mice were used in each group (5 male and 5 female), except in the high dose groups for the BiP-SGSH-vIGF2 construct and the BiP-SGSH (stabilized) construct. Due to breeding issues and timeline constraints, only five male mice and zero female mice were included in each of these two groups. The “stabilized” SGSH constructs refer to the sequences containing the A482Y and E488V double mutation. The SGSH activity assays and GAG reduction assays were performed using tissue homogenates for brain, spinal cord, and liver.

In an organism lacking sufficient SGSH activity, long linear polysaccharides called GAGs accumulate in lysosomes. Heparan sulfate (HS) is a type of GAG that is a substrate for SGSH. Measurement of HS levels in different tissues reveals whether SGSH is functioning to desulfate HIS so that it no longer accumulates in the lysosome. HIS levels are a standard biomarker in MPSIIIA research.

To measure HS levels in the tissues tested, the HS is chemically broken down and quantified by LC-MS/MS. Acid and heat hydrolysis and butanolysis derivatization of HIS results in (Gl/Id) uronic-glucosamine disaccharide derivatives with butane groups on both the carboxylic acid and reducing end of each isomer. The resulting products can be quantified by LC-MS/MS SRM (Selected Reaction Monitoring) experiments.

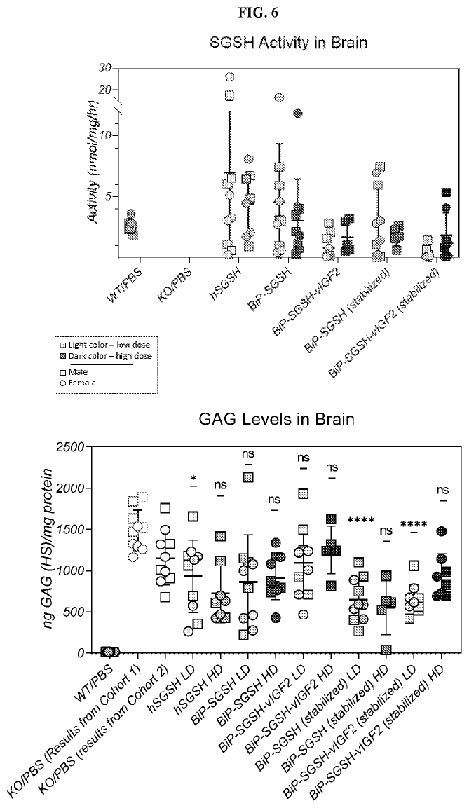

FIG. 6 shows SGSH activity and GAG (HS) levels in the brains of the mice in the study. No SGSH activity is detected in the PBS control KO mice. The group treated with hSGSH has high levels of SGSH activity in both the high dose and the low dose group; SGSH activity does not increase in the high dose group compared with the low dose group. The mice treated with vIGF2-tagged constructs have lower SGSH activity in the brain than the mice treated with untagged constructs. The mice treated with stabilized SGSH do not have higher SGSH activity than those treated with non-stabilized SGSH.

In the analysis of GAG levels in the brain, GAG levels in treated KO mice were compared against levels in PBS control KO mice. Brown-Forsythe and Welch ANOVA tests were performed, with Dunnett's T3 multiple comparisons test, to assess significance in the reduction of GAG levels. The set of PBS control KO samples was assayed twice: once together with the set of low dose treated samples (i.e., Cohort 1), and once together with the set of high dose treated samples (i.e., Cohort 2). GAG level measurements for the treated groups were compared against the PBS control data set from the appropriate Cohort.

GAG reduction appears to have been most effective for the groups treated with the stabilized BiP-SGSH construct as well as the stabilized BiP-SGSH-vIGF2 construct at the low dose (p<0.0001). There was also a significant (p=0.035) decrease in GAG levels for the group treated with hSGSH at the low dose.

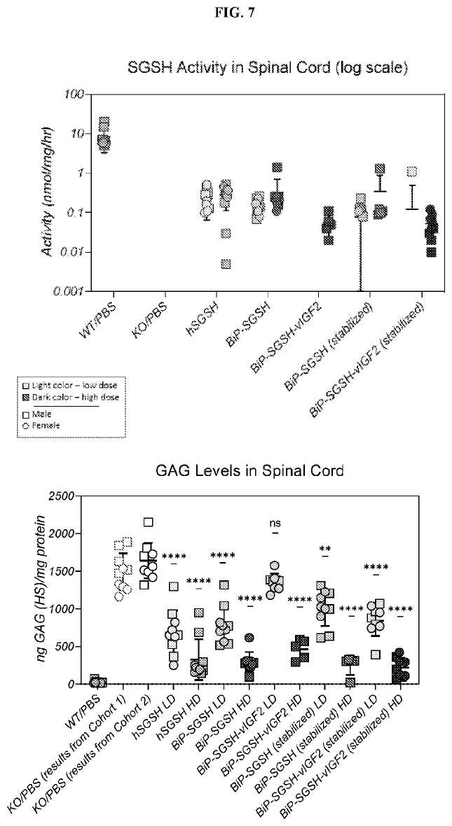

FIG. 7 shows SGSH activity and GAG (HS) levels in the spinal cords of the mice in the study. No SGSH activity is detected in the PBS control KO mice. None of the treatment groups have SGSH activity as high as the WT control mice. The mice treated with vIGF2-tagged constructs show little or no measurable SGSH activity at the low dose but do have measurable activity at the high dose.

In the analysis of GAG levels in the spinal cord, GAG levels in treated KO mice were compared against levels in PBS control KO mice. Brown-Forsythe and Welch ANOVA tests were performed, with Dunnett's T3 multiple comparisons test, to assess significance in the reduction of GAG levels. The set of PBS control KO samples was assayed twice: once together with the set of low dose treated samples (i.e., Cohort 1), and once together with the set of high dose treated samples (i.e., Cohort 2). GAG level measurements for the treated groups were compared against the PBS control data set from the appropriate Cohort.

At the low dose, GAG reduction appears to have been most effective for the groups treated with hSGSH, BiP-SGSH, and BiP-SGSH-vIGF2 (stabilized). At the high dose, all groups showed significant (p<0.0001) reduction in GAG levels.

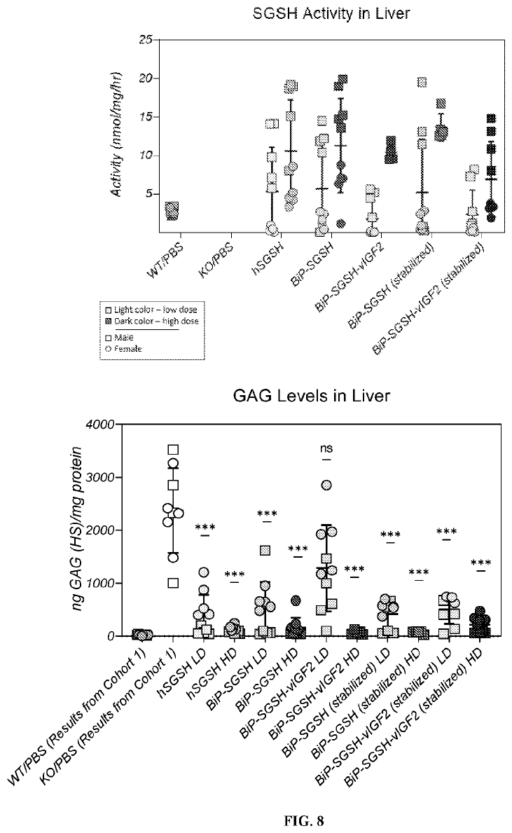

FIG. 8 shows SGSH activity and GAG (HS) levels in the livers of the mice in the study. No SGSH activity is detected in the PBS control KO mice. A higher average activity measurement is seen for the high dose treatment groups than for the low dose treatment groups. The highest average activity is seen in the groups treated with hSGSH and with BiP-SGSH.

In the analysis of GAG levels in the liver, GAG levels in treated KO mice were compared against levels in PBS control KO mice Brown-Forsythe and Welch ANOVA tests were performed, with Dunnett's T3 multiple comparisons test, to assess significance in the reduction of GAG levels. All treatment groups showed a significant reduction in GAG levels (p<0.001) except for the group treated with low dose BiP-SGSH-vIGF2.

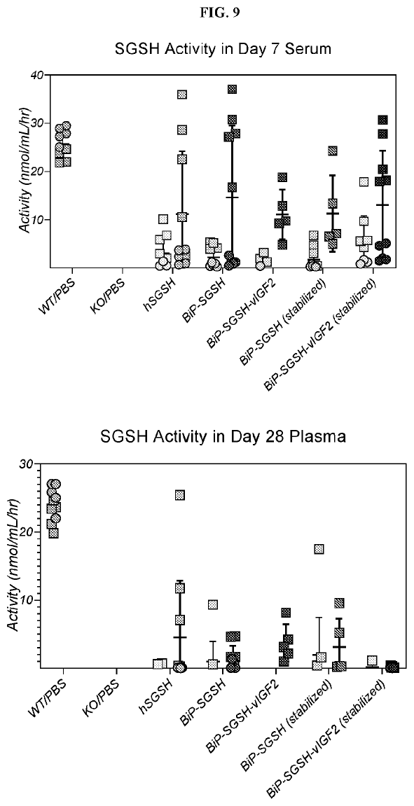

FIG. 9 shows SGSH activity in the serum (7 days post-treatment) and plasma (terminal bleed 28 days post-treatment) of mice in the study. No SGSH activity is detected in the PBS control KO mice. Activity levels are overall higher in the high dose groups than the low dose groups, and considerably lower in the plasma at day 28 than in the serum at day 7, suggesting a possible antibody response.

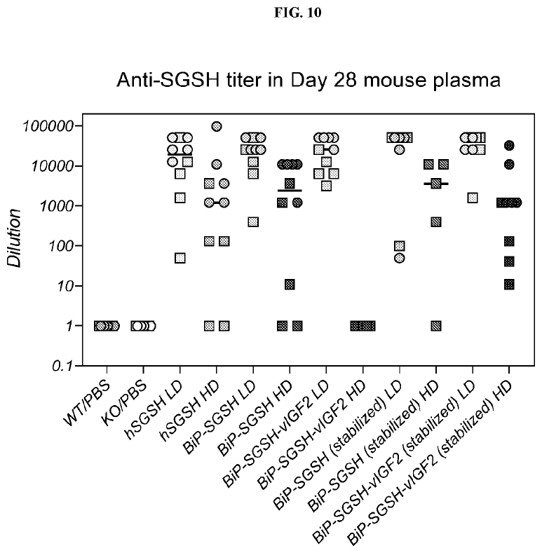

An ELISA was performed in order to determine the anti-SGSH antibody titer. Plates were first coated with purified SGSH protein, then serial dilutions of plasma were applied to the coated plates. After washing the plate, a peroxidase-conjugated secondary antibody was added to the plates and the plate was washed again. Color development after addition of peroxidase substrate indicated the presence of anti-SGSH antibodies in the plasma, and an end-point titer was calculated from the results (FIG. 10). Overall, higher titers were seen for the low dose groups.

Based on these studies, WT hSGSH has the highest expression (based on activity) in mice, but BiP-SGSH (stabilized) and BiP-SGSH-vIGF2 (stabilized) decrease GAG levels in mouse tissues more effectively than hSGSH. BiP-SGSH-vIGF2 (stabilized) appeared to decrease GAG levels slightly more effectively than the untagged BiP-SGSH (stabilized).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### NUMBERED EMBODIMENTS

Notwithstanding the appended claims, the disclosure sets forth the following numbered embodiments:

1. A variant human N-sulfoglucosamine sulfohydrolase (SGSH) protein, wherein the variant human SGSH protein comprises at least one mutation selected from A482Y and F488V compared to SEQ ID NO:1, and wherein the native signal peptide corresponding to amino acid residues 1-20 of SEQ ID NO: 1 is replaced with a signal peptide comprising a sequence selected from the group consisting of SEQ ID NOs: 22-27.

2. The variant human SGSH protein of embodiment 1, wherein the variant SGSH protein comprises the A482Y mutation.

3. The variant human SGSH protein of embodiment 1, wherein the variant SGSH protein comprises the E488V mutation.

4. The variant human SGSH protein of embodiment 1, wherein the variant SGSH protein comprises the A482Y mutation and the E488V mutation.

5. The variant human SGSH protein of embodiment 2, wherein the variant SGSH protein comprises an amino acid sequence that is at least 95% identical to SEQ ID NO: 2.

6. The variant human SGSH protein of embodiment 5, wherein the variant SGSH protein comprises an amino acid sequence that is at least 98% identical to SEQ ID NO: 2.

7. The variant human SGSH protein of embodiment 6, wherein the variant SGSH protein comprises the amino acid sequence of SEQ ID NO: 2.

8. The variant human SGSH protein of embodiment 3, wherein the variant SGSH protein comprises an amino acid sequence that is at least 95% identical to SEQ ID NO: 3.

9. The variant human SGSH protein of embodiment 8, wherein the variant SGSH protein comprises an amino acid sequence that is at least 98% identical to SEQ ID NO: 3.

10. The variant human SGSH protein of embodiment 9, wherein the variant SGSH protein comprises the amino acid sequence of SEQ ID NO: 3.

11. The variant human SGSH protein of embodiment 4, wherein the variant SGSH protein comprises an amino acid sequence that is at least 95% identical to SEQ ID NO: 4.

12. The variant human SGSH protein of embodiment 11, wherein the variant SGSH protein comprises an amino acid sequence that is at least 98% identical to SEQ ID NO: 4.

13. The variant human SGSH protein of embodiment 12, wherein the variant SGSH protein comprises the amino acid sequence of SEQ ID NO: 4.

14. The variant human SGSH protein of any one of embodiments 1 to 13, wherein the signal peptide is a non-native signal peptide comprising an amino acid sequence selected from the group consisting of SEQ ID NOs: 22-27.

15. The variant human SGSH protein of any one of embodiments 1 to 14, wherein the variant human SGSH protein further comprises a lysosomal cleavage peptide.

16. The variant human SGSH protein of any one of embodiments 1 to 15, further comprising a variant insulin-like growth factor 2 (vIGF2) peptide.

17. The variant human SGSH protein of embodiment 16, wherein the vIGF2 peptide is N terminal to the variant human SGSH protein.

18. The variant human SGSH protein of embodiment 16, wherein the vIGF2 peptide is C terminal to the variant human SGSH protein.

19. The variant human SGSH protein of any one of embodiments 16 to 18, wherein the vIGF2 peptide comprises an amino acid sequence selected from the group consisting of SEQ ID NOs: 29-91.

20. The variant human SGSH protein of any one of embodiments 16 to 18, wherein the vIGF2 peptide comprises an amino acid sequence that is at least 98% identical to a SEQ ID NO:40 or SEQ ID NO:41.

21. A nucleic acid construct encoding the variant human SGSH protein of any one of embodiments 1 to 20.

22. A gene therapy vector comprising the nucleic acid construct of embodiment 21.

23. The gene therapy vector of embodiment 22, wherein the gene therapy vector is a virus vector.

24. The gene therapy vector of embodiment 23, wherein the virus vector is an adenovirus vector, an adeno-associated virus (AAV) vector, a retrovirus vector, a lentivirus vector, a pox virus vector, a vaccinia virus vector, an adenovirus vector, or a herpes virus vector.

25. The gene therapy vector of embodiment 24, wherein the AAV vector is an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAV-PHP.B vector, an AAVhu68 vector, an AAV-DJ vector, an AAV2/9 vector, a TM-AAV6 vector, an AAV-PHP.A vector, an AAV-PHP.S vector or an AAV-PHPeB vector.

26. The nucleic acid construct of embodiment 21, wherein the nucleic acid construct is a plasmid.

27. A pharmaceutical composition comprising a therapeutically effective amount of the nucleic acid construct of any one of embodiments 21 to 26, or the gene therapy vector of any one of embodiments 22 to 25, and a pharmaceutically acceptable carrier or excipient.

28. The pharmaceutical composition of embodiment 27, wherein the excipient comprises a non-ionic, low-osmolar compound, a buffer, a polymer, a salt, or a combination thereof.

29. A method for treating Mucopolysaccharidosis IIIA (MPS IIIA) comprising administering to a subject in need thereof the pharmaceutical composition of embodiment 27 or embodiment 28.

30. The method of embodiment 29, wherein the administering is performed intrathecally, intraocularly, intravitreally, retinally, intravenously, intramuscularly, intraventricularly, intracerebrally, intracerebellarly, intracerebroventricularly, intraperenchymally, subcutaneously, intradermally or topically, or via a combination thereof.

31. The nucleic acid construct of any one of embodiments 21 to 26, or the gene therapy vector of any one of embodiments 22 to 25 for use in preparation of a medicament for treating MPS IIIA.

32. A pharmaceutical composition comprising the variant human SGSH protein of any one of embodiments 1 to 20 and a pharmaceutically acceptable carrier or excipient.

33. A method of treating MPS IIIA, comprising administering the pharmaceutical composition of embodiment 33 to a subject in need thereof.

34. The method of embodiment 33, wherein the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

35. The method of any one of embodiments 29-30, 33 and 34, wherein administering the pharmaceutical composition prevents, reduces or reverses accumulation of heparan sulfate in the subject.

What is claimed is:

1. A variant human N-sulfoglucosamine sulfohydrolase (SGSH) protein, wherein the variant human SGSH protein comprises at least one mutation selected from A482Y and E488V compared to SEQ ID NO:1, and wherein the native signal peptide corresponding to amino acid residues 1-20 of SEQ ID NO:1 is replaced with a signal peptide comprising a sequence selected from the group consisting of SEQ ID NOs: 22-27.

2. The variant human SGSH protein of claim 1, wherein the variant SGSH protein comprises the A482Y mutation.

3. The variant human SGSH protein of claim 1, wherein the variant SGSH protein comprises the E488V mutation.

4. The variant human SGSH protein of claim 1, wherein the variant SGSH protein comprises the A482Y mutation and the E488V mutation.

5. The variant human SGSH protein of claim 2, wherein the variant SGSH protein comprises an amino acid sequence that is at least 95% identical to SEQ ID NO: 2.

6. The variant human SGSH protein of claim 5, wherein the variant SGSH protein comprises an amino acid sequence that is at least 98% identical to SEQ ID NO: 2.

7. The variant human SGSH protein of claim 6, wherein the variant SGSH protein comprises the amino acid sequence of SEQ ID NO: 2.

8. The variant human SGSH protein of claim 3, wherein the variant SGSH protein comprises an amino acid sequence that is at least 95% identical to SEQ ID NO: 3.

9. The variant human SGSH protein of claim 8, wherein the variant SGSH protein comprises an amino acid sequence that is at least 98% identical to SEQ ID NO: 3.

10. The variant human SGSH protein of claim 9, wherein the variant SGSH protein comprises the amino acid sequence of SEQ ID NO: 3.

11. The variant human SGSH protein of claim 4, wherein the variant SGSH protein comprises an amino acid sequence that is at least 95% identical to SEQ ID NO: 4.

12. The variant human SGSH protein of claim 11, wherein the variant SGSH protein comprises an amino acid sequence that is at least 98% identical to SEQ ID NO: 4.

13. The variant human SGSH protein of claim 12, wherein the variant SGSH protein comprises the amino acid sequence of SEQ ID NO: 4.

14. The variant human SGSH protein of any one of claims 1 to 13, wherein the signal peptide is a non-native signal peptide comprising an amino acid sequence selected from the group consisting of SEQ ID NOs: 22-27.

15. The variant human SGSH protein of any one of claims 1 to 14, wherein the variant human SGSH protein further comprises a lysosomal cleavage peptide.

16. The variant human SGSH protein of any one of claims 1 to 15, further comprising a variant insulin-like growth factor 2 (vIGF2) peptide.

17. The variant human SGSH protein of claim 16, wherein the vIGF2 peptide is N terminal to the variant human SGSH protein.

18. The variant human SGSH protein of claim 16, wherein the vIGF2 peptide is C terminal to the variant human SGSH protein.

19. The variant human SGSH protein of any one of claims 16 to 18, wherein the vIGF2 peptide comprises an amino acid sequence selected from the group consisting of SEQ ID NOs: 29-91.

20. The variant human SGSH protein of any one of claims 16 to 18, wherein the vIGF2 peptide comprises an amino acid sequence that is at least 98% identical to a SEQ ID NO:40 or SEQ ID NO:41.

21. A nucleic acid construct encoding the variant human SGSH protein of any one of claims 1 to 20.

22. A gene therapy vector comprising the nucleic acid construct of claim 21.

23. The gene therapy vector of claim 22, wherein the gene therapy vector is a virus vector.

24. The gene therapy vector of claim 23, wherein the virus vector is an adenovirus vector, an adeno-associated virus (AAV) vector, a retrovirus vector, a lentivirus vector, a pox virus vector, a vaccinia virus vector, an adenovirus vector, or a herpes virus vector.

25. The gene therapy vector of claim 24, wherein the AAV vector is an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAV-PHP.B vector, an AAVhu68 vector, an AAV-DJ vector, an AAV2/9 vector, a TM-AAV6 vector, an AAV-PHP.A vector, an AAV-PHP.S vector or an AAV-PHPeB vector.

26. The nucleic acid construct of claim 21, wherein the nucleic acid construct is a plasmid.

27. A pharmaceutical composition comprising a therapeutically effective amount of the nucleic acid construct of any one of claims 21 to 26, or the gene therapy vector of any one of claims 22 to 25, and a pharmaceutically acceptable carrier or excipient.

28. The pharmaceutical composition of claim 27, wherein the excipient comprises a non-ionic, low-osmolar compound, a buffer, a polymer, a salt, or a combination thereof.

29. A method for treating Mucopolysaccharidosis IIIA (MPS IIIA) comprising administering to a subject in need thereof the pharmaceutical composition of claim 27 or claim 28.

30. The method of claim 29, wherein the administering is performed intrathecally, intraocularly, intravitreally, retinally, intravenously, intramuscularly, intraventricularly, intracerebrally, intracerebellarly, intracerebroventricularly, intraperenchymally, subcutaneously, intradermally or topically, or via a combination thereof.

31. The nucleic acid construct of any one of claims 21 to 26, or the gene therapy vector of any one of claims 22 to 25 for use in preparation of a medicament for treating MPS IIIA.

32. A pharmaceutical composition comprising the variant human SGSH protein of any one of claims 1 to 20 and a pharmaceutically acceptable carrier or excipient.

33. A method of treating MPS IIIA, comprising administering the pharmaceutical composition of claim 33 to a subject in need thereof.

34. The method of claim 33, wherein the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

35. The method of any one of claims 29-30, 33 and 34, wherein administering the pharmaceutical composition prevents, reduces or reverses accumulation of heparan sulfate in the subject.


SEQUENCE LISTING (96 sequences)

SEQ ID NO: 1 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 2 - protein, 502 aa, synthetic construct
  REGION 1..502: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLEEKLSPQCQPLHNEL

SEQ ID NO: 3 - protein, 502 aa, synthetic construct
  REGION 1..502: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLVEKLSPQCQPLHNEL

SEQ ID NO: 4 - protein, 502 aa, synthetic construct
  REGION 1..502: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNEL

SEQ ID NO: 5 - protein, 500 aa, synthetic construct
  REGION 1..500: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 6 - protein, 500 aa, synthetic construct
  REGION 1..500: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLEEKLSPQCQPLHNEL

SEQ ID NO: 7 - protein, 500 aa, synthetic construct
  REGION 1..500: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLVEKLSPQCQPLHNEL

SEQ ID NO: 8 - protein, 500 aa, synthetic construct
  REGION 1..500: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNEL

SEQ ID NO: 9 - protein, 581 aa, synthetic construct
  REGION 1..581: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 10 - protein, 583 aa, synthetic construct
  REGION 1..583: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 11 - protein, 583 aa, synthetic construct
  REGION 1..583: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLEEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 12 - protein, 583 aa, synthetic construct
  REGION 1..583: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 13 - protein, 583 aa, synthetic construct
  REGION 1..583: note = SGSH variant protein
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 14 - protein, 581 aa, synthetic construct
  REGION 1..581: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLEEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 15 - protein, 581 aa, synthetic construct
  REGION 1..581: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 16 - protein, 581 aa, synthetic construct
  REGION 1..581: note = SGSH variant protein
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 17 - protein, 482 aa, synthetic construct
  REGION 1..482: note = SGSH variant protein
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 18 - protein, 482 aa, synthetic construct
  REGION 1..482: note = SGSH variant protein
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLEEKLSPQCQPLHNEL

SEQ ID NO: 19 - protein, 482 aa, synthetic construct
  REGION 1..482: note = SGSH variant protein
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLVEKLSPQCQPLHNEL

SEQ ID NO: 20 - protein, 482 aa, synthetic construct
  REGION 1..482: note = SGSH variant protein
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNEL

SEQ ID NO: 21 - protein, 20 aa, Homo sapiens
MSCPVPACCALLLVLGLCRA

SEQ ID NO: 22 - protein, 18 aa, Homo sapiens
MKLSLVAAMLLLLSAARA

SEQ ID NO: 23 - protein, 28 aa, synthetic construct
  REGION 1..28: note = Signal Peptide Sequence
MKLSLVAAMLLLLSLVAAMLLLLSAARA

SEQ ID NO: 24 - protein, 25 aa, synthetic construct
  REGION 1..25: note = Signal Peptide Sequence
MKLSLVAAMLLLLWVALLLLSAARA

SEQ ID NO: 25 - protein, 26 aa, synthetic construct
  REGION 1..26: note = Signal Peptide Sequence
MKLSLVAAMLLLLSLVALLLLSAARA

SEQ ID NO: 26 - protein, 26 aa, synthetic construct
  REGION 1..26: note = Signal Peptide Sequence
MKLSLVAAMLLLLALVALLLLSAARA

SEQ ID NO: 27 - protein, 17 aa, synthetic construct
  REGION 1..17: note = Signal Peptide Sequence
MGVKVLFALICIAVAEA

SEQ ID NO: 28 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 29 - protein, 67 aa, synthetic construct
  REGION 1..67: note = IGF2 variant
AYRPSETLCGGELVDTLQFVCGDRGSYFSRPASRVSRRSRGIVEECCFRSCDLALLETYCATPAKSE

SEQ ID NO: 30 - protein, 67 aa, synthetic construct
  REGION 1..67: note = IGF2 variant
AYRPSETLCGGELVDTLQFVCGDRGFLFSRPASRVSRRSRGIVEECCFRSCDLALLETYCATPAKSE

SEQ ID NO: 31 - protein, 67 aa, synthetic construct
  REGION 1..67: note = IGF2 variant
AYRPSETLCGGELVDTLQFVCGDRGFYFSRPASRVSRRSRGILEECCFRSCDLALLETYCATPAKSE

SEQ ID NO: 32 - protein, 67 aa, synthetic construct
  REGION 1..67: note = IGF2 variant
AYRPSETLCGGELVDTLQFVCGDRGFYFSRPASRVSRRSRGIVEECCTRSCDLALLETYCATPAKSE

SEQ ID NO: 33 - protein, 67 aa, synthetic construct
  REGION 1..67: note = IGF2 variant
AYRPSETLCGGELVDTLQFVCGDRGFYFSRPASRVSRRSRGIVEECCFSSCDLALLETYCATPAKSE

SEQ ID NO: 34 - protein, 67 aa, synthetic construct
  REGION 1..67: note = IGF2 variant
AYRPSETLCGGELVDTLQFVCGDRGFYFSRPASRVSRRSRGIVEECCFRICDLALLETYCATPAKSE

SEQ ID NO: 35 - protein, 67 aa, synthetic construct
  REGION 1..67: note = IGF2 variant
AYRPSETLCGGELVDTLQFVCGDRGFYFSRPASRVSRRSRGIVEECCFRSCDLRLLETYCATPAKSE

SEQ ID NO: 36 - protein, 67 aa, synthetic construct
  REGION 1..67: note = IGF2 variant
AYRPSETLCGGELVDTLQFVCGDRGFYFSRPASRVSRRSRGIVEECCFRSCDLARLETYCATPAKSE

SEQ ID NO: 37 - protein, 67 aa, synthetic construct
  REGION 1..67: note = IGF2 variant
AYRPSETLCGGELVDTLQFVCGDRGSLFSRPASRVSRRSRGILEECCTSICDLRRLETYCATPAKSE

SEQ ID NO: 38 - protein, 61 aa, synthetic construct
  REGION 1..61: note = IGF2 variant
TLCGGELVDTLQFVCGDRGFLFSRPASRVSRRSRGIVEECCFRSCDLALLETYCATPARSE

SEQ ID NO: 39 - protein, 60 aa, synthetic construct
  REGION 1..60: note = IGF2 variant
LCGGELVDTLQFVCGDRGFLFSRPASRVSRRSRGIVEECCFRSCDLALLETYCATPARSE

SEQ ID NO: 40 - protein, 63 aa, synthetic construct
  REGION 1..63: note = IGF2 variant
SRTLCGGELVDTLQFVCGDRGFLFSRPASRVSRRSRGIVEECCFRSCDLALLETYCATPARSE

SEQ ID NO: 41 - protein, 63 aa, synthetic construct
  REGION 1..63: note = IGF2 variant
SRTLCGGELVDTLQFVCGDRGFLFSRPASRVSRRSRGIVEECCFRSCDLALLETYCATPAKSE

SEQ ID NO: 42 - protein, 67 aa, synthetic construct
  REGION 1..67: note = IGF2 variant
AYRPSRTLCGGELVDTLQFVCGDRGFYFSRPASRVSRRSRGIVEECCFRSCDLALLETYCATPAKSE

SEQ ID NO: 43 - protein, 63 aa, synthetic construct
  REGION 1..63: note = IGF2 variant
SRTLCGGELVDTLQFVCGDRGFLFSRPASRVSRRSRGIVEECCFRECDLALLETYCATPARSE

SEQ ID NO: 44 - protein, 18 aa, synthetic construct
  REGION 1..18: note = IGF2 variant
GGGGSGGGGSRPRAVPTQ

SEQ ID NO: 45 - protein, 25 aa, synthetic construct
  REGION 1..25: note = IGF2 variant
YIPAKQGLQGAQMGQPGGGGSGGGG

SEQ ID NO: 46 - protein, 18 aa, synthetic construct
  REGION 1..18: note = IGF2 variant
RPRAVPTQGGSGSGSTSS

SEQ ID NO: 47 - protein, 81 aa, synthetic construct
  REGION 1..81: note = IGF2 variant
SRTLCGGELVDTLQFVCGDRGFLFSRPASRVSRRSRGIVEECCFRSCDLALLETYCATPARSEGGGGSGGGGSRPRAVPTQ

SEQ ID NO: 48 - protein, 88 aa, synthetic construct
  REGION 1..88: note = IGF2 variant
YIPAKQGLQGAQMGQPGGGGSGGGGSRTLCGGELVDTLQFVCGDRGFLFSRPASRVSRRSRGIVEECCFRSCDLALLETYCATPARSE

SEQ ID NO: 49 - protein, 81 aa, synthetic construct
  REGION 1..81: note = IGF2 variant
RPRAVPTQGGSGSGSTSSSRTLCGGELVDTLQFVCGDRGFLFSRPASRVSRRSRGIVEECCFRECDLALLETYCATPARSE

SEQ ID NO: 50 - protein, 63 aa, synthetic construct
  REGION 1..63: note = IGF2 variant
SRTLCGGELVDTNQFVCGDRGFLFSRGASRVSRGSRGIVEWCCFRGCDLALLETYCATPMRGE

SEQ ID NO: 51 - protein, 63 aa, synthetic construct
  REGION 1..63: note = IGF2 variant
SRTLCGGELVDTLQFVCGDRGFLFSRGASRVSRGSRGIVEWCCFRGCDLALLETYCATPMRGE

SEQ ID NO: 52 - protein, 63 aa, synthetic construct
  REGION 1..63: note = IGF2 variant
SRTLCGGELVDTNQFVCGDRGFLFSRGASRVSRGSRGIVEECCFRGCDLALLETYCATPMRGE

SEQ ID NO: 53 - protein, 53 aa, synthetic construct
  REGION 1..53: note = IGF2 variant
SRTLCGGELVDTLQFVCGDRGFLFSRPIVEECCFRSCDLALLETYCATPARSE

SEQ ID NO: 54 - protein, 53 aa, synthetic construct
  REGION 1..53: note = IGF2 variant
SRTLCGGELVDTLQFVCGDRGFLFSRGIVEECCFRSCDLALLETYCATPARSE

SEQ ID NO: 55 - protein, 55 aa, synthetic construct
  REGION 1..55: note = IGF2 variant
SRTLCGGELVDTLQFVCGDRGFLFSRPAGIVEECCFRSCDLALLETYCATPARSE

SEQ ID NO: 56 - protein, 53 aa, synthetic construct
  REGION 1..53: note = IGF2 variant
SRTLCGGELDDTLQFVCGDRGALRSRGIDEECCFRSCDLALLETYCATPARSE

SEQ ID NO: 57 - protein, 63 aa, synthetic construct
  REGION 1..63: note = IGF2 variant
SRTLCGGELVDTLQFVCGRRGELFSRPASRVSRRSRGIVEECCFRECDLALLETYCATPARSE

SEQ ID NO: 58 - protein, 55 aa, synthetic construct
  REGION 1..55: note = IGF2 variant
SRTLCGGELVDTLQFVCGRRGELFSRPAGIVEECCFRECDLALLETYCATPARSE

SEQ ID NO: 59 - protein, 63 aa, synthetic construct
  REGION 1..63: note = IGF2 variant
SRTLCGGELVDTLQFVCGRRGFLFSRPASRVSRRSRGIVEECCFRSCDLALLETYCATPARSE

SEQ ID NO: 60 - protein, 63 aa, synthetic construct
  REGION 1..63: note = IGF2 variant
SRTLCGGELVDTLQWVCGDRGFLFSRPASRVSRRSRGIVEECCFRSCDLALLETYCATPARSE

SEQ ID NO: 61 - protein, 63 aa, synthetic construct
  REGION 1..63: note = IGF2 variant
SRTLCGGELVDWLQFVCGDRGFLFSRPASRVSRRSRGIVEECCFRSCDLALLETYCATPARSE

SEQ ID NO: 62 - protein, 63 aa, synthetic construct
  REGION 1..63: note = IGF2 variant
SRTLCGGELVDTLQFVCGKRGFLFSRPASRVSRRSRGIVEECCFRSCDLALLETYCATPARSE

SEQ ID NO: 63 - protein, 63 aa, synthetic construct
  REGION 1..63: note = IGF2 variant
SRTLCGGELVDYLQFVCGDRGFLFSRPASRVSRRSRGIVEECCFRSCDLALLETYCATPARSE

SEQ ID NO: 64 - protein, 63 aa, synthetic construct
  REGION 1..63: note = IGF2 variant
SRTLCGGELVDTLQFVCGDRGELFSRPASRVSRRSRGIVEECCFRSCDLALLETYCATPARSE

SEQ ID NO: 65 - protein, 63 aa, synthetic construct
  REGION 1..63: note = IGF2 variant
SRTLCGGELVDVLQFVCGDRGFLFSRPASRVSRRSRGIVEECCFRSCDLALLETYCATPARSE

SEQ ID NO: 66 - protein, 63 aa, synthetic construct; identical to SEQ ID NO: 43
  REGION 1..63: note = IGF2 variant

SEQ ID NO: 67 - protein, 63 aa, synthetic construct
  REGION 1..63: note = IGF2 variant
SRTLCGGELVDTLQFVCGDRGFLFSRPASRVSRRSRGIVEECCFRDCDLALLETYCATPARSE

SEQ ID NO: 68 - protein, 63 aa, synthetic construct
  REGION 1..63: note = IGF2 variant
SRTLCGGELVDTLQFVCGDRGSLFSRPASRVSRRSRGILEECCFRSCDLALLETYCATPARSE

SEQ ID NO: 69 - protein, 63 aa, synthetic construct
  REGION 1..63: note = IGF2 variant
SRTLCGGELVDTLQFVCGDRGFLFSRPASRVSRRSRGILEECCFRSCDLALLETYCATPARSE

SEQ ID NO: 70 - protein, 63 aa, synthetic construct
  REGION 1..63: note = IGF2 variant
SRTLCGGELEDTLQFVCGDRGSLRSRPASRVSRRSRGIEEECCFRSCDLALLETYCATPARSE

SEQ ID NO: 71 - protein, 59 aa, synthetic construct
  REGION 1..59: note = IGF2 variant
SRTLCGGELVDTLQFVCGDRGFLFSRGGGGSRGIVEECCFRSCDLALLETYCATPARSE

SEQ ID NO: 72 - protein, 56 aa, synthetic construct
  REGION 1..56: note = IGF2 variant
SRTLCGGELVDTLQFVCGDRGFLFSGGGSGIVEECCFRSCDLALLETYCATPARSE

SEQ ID NO: 73 - protein, 59 aa, synthetic construct
  REGION 1..59: note = IGF2 variant
SRTLCGGELVDTLQFVCGDRGFLFSRGGGGSRGILEECCFRSCDLALLETYCATPARSE

SEQ ID NO: 74 - protein, 63 aa, synthetic construct
  REGION 1..63: note = IGF2 variant
SRTACGGELVDTLQFVCGDRGFLFSRPASRVSRRSRGIVEECCFRSCDLALLETYCATPARSE

SEQ ID NO: 75 - protein, 63 aa, synthetic construct
  REGION 1..63: note = IGF2 variant
SQAACGGELVDTLQFVCGDRGFLFSRPASRVSRRSRGIVEECCFRSCDLALLETYCATPARSE

SEQ ID NO: 76 - protein, 63 aa, synthetic construct
  REGION 1..63: note = IGF2 variant
SRTLCGGELVDTLQFVCGDEGFLFSRPASEVSEESRGIVEECCFRSCDLALLETYCATPARSE

SEQ ID NO: 77 - protein, 63 aa, synthetic construct
  REGION 1..63: note = IGF2 variant
SRTLCGGELVDTLQFVCGDEGFLFSRPASEVSRRSRGIVEECCFRSCDLALLETYCATPARSE

SEQ ID NO: 78 - protein, 63 aa, synthetic construct
  REGION 1..63: note = IGF2 variant
SRTLCGGELVDTLQFVCGRRGFLFSRPASRVSRRSRGIVEECCFRDCDLALLETYCATPARSE

SEQ ID NO: 79 - protein, 63 aa, synthetic construct
  REGION 1..63: note = IGF2 variant
SRTLCGGELVDVLQFVCGRRGFLFSRPASRVSRRSRGIVEECCFRDCDLALLETYCATPARSE

SEQ ID NO: 80 - protein, 59 aa, synthetic construct
  REGION 1..59: note = IGF2 variant
SRTLCGGELVDTLQFVCGDRGFLFSRGGGGSRGILEECCFRDCDLALLETYCATPARSE

SEQ ID NO: 81 - protein, 59 aa, synthetic construct
  REGION 1..59: note = IGF2 variant
SRTLCGGELVDTLQFVCGDRGFLFSRGGGGSRGILEECCFRECDLALLETYCATPARSE

SEQ ID NO: 82 - protein, 67 aa, synthetic construct
  REGION 1..67: note = IGF2 variant
SRTLCGGELVDTLQFVCGDRGFLFRLPSRPVSRHSHRRSRGIVEECCFQRCNLALLETYCATPARSE

SEQ ID NO: 83 - protein, 67 aa, synthetic construct
  REGION 1..67: note = IGF2 variant
SRTLCGGELVDTLQFVCGDRGFLFRLPSRPVSRHSHRRSRGILEECCFQECNLALLETYCATPARSE

SEQ ID NO: 84 - protein, 63 aa, synthetic construct
  REGION 1..63: note = IGF2 variant
SRTLCGGELVDTLQFVCGDRGFLFSRPASRVSRGSRGIVEECCFRSCDLALLETYCATPARSE

SEQ ID NO: 85 - protein, 63 aa, synthetic construct
  REGION 1..63: note = IGF2 variant
SRTLCGGELVDTLQFVCGDRGFLFSRPASRVSRGSRGIVEWCCFRSCDLALLETYCATPARSE

SEQ ID NO: 86 - protein, 63 aa, synthetic construct
  REGION 1..63: note = IGF2 variant
SRTLCGGELVDTLQFVCGDRGFLFSRPASRVSRGSRGIVEWCCFRGCDLALLETYCATPARSE

SEQ ID NO: 87 - protein, 63 aa, synthetic construct
  REGION 1..63: note = IGF2 variant
SRTLCGGELVDTLQFVCGDRGFLFSRGASRVSRGSRGIVEWCCFRGCDLALLETYCATPARSE

SEQ ID NO: 88 - protein, 63 aa, synthetic construct
  REGION 1..63: note = IGF2 variant
SRTLCGGELVDTNQFVCGDRGFLFSRGASRVSRGSRGIVEWCCFRGCDLALLETYCATPARSE

SEQ ID NO: 89 - protein, 63 aa, synthetic construct
  REGION 1..63: note = IGF2 variant
SRTLCGGELVDTNQFVCGDRGFLFSRGASRVSRGSRGIVEWCCFRGCDLALLETYCATPARGE

SEQ ID NO: 90 - protein, 63 aa, synthetic construct; identical to SEQ ID NO: 50
  REGION 1..63: note = IGF2 variant

SEQ ID NO: 91 - protein, 63 aa, synthetic construct
  REGION 1..63: note = IGF2 variant
LRTLCGGELVDTNQFVCGDRGFLFSRGASRVSRGSRGIVEWCCFRGCDLALLETYCATPMRGE

SEQ ID NO: 92-93 - skipped (2 entries)

SEQ ID NO: 94 - RNA, 13 nt, unidentified
  misc_feature 1..13: note = Kozak sequence
  source 1..13: mol_type = other RNA
gccgccaccatgg

SEQ ID NO: 95-96 - skipped (2 entries)